(19) United States
(12) Patent Application Publication
(10) Pub. No.: US 2006/0009510 A1
(43) Pub. Date: Jan. 12, 2006

(54) Method of synthesizing indolinone compounds

(75) Inventors: Jeffrey L. Havens, Mattawan, MI (US); Rajappa Vaidyanathan, Portage, MI (US)

Correspondence Address: AGOURON PHARMACEUTICALS, INC., 10777 SCIENCE CENTER DRIVE, SAN DIEGO, CA, 92121, US

(73) Assignee: Pharmacia & Upjohn Company LLC

(21) Appl. No.: 11/174,892

(22) Filed: Jul. 5, 2005

Related U.S. Application Data
(60) Provisional application No. 60/586,865, filed on Jul. 9, 2004.

(51) Int. Cl. C07D 209/36; A61K 31/404

(52) U.S. Cl. 514/418; 548/484

(57) ABSTRACT

Disclosed are methods of preparing pyrrole compounds of formula 14 and indolinone compounds of formula 1 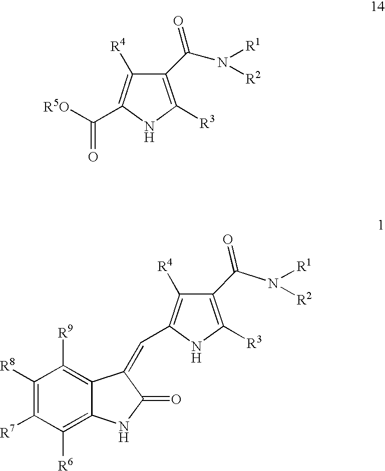 via a synthetic route wherein the amide sidechain on the pyrrole moiety is attached prior to pyrrole formation. The compounds 14 produced by the methods herein are useful in the synthesis of compounds of formula 1, which are useful in the treatment of abnormal cell growth, such as cancer.

This application claims the benefit of U.S. Provisional Application Ser. No. 60/586,865, filed Jul. 9, 2004, the disclosure of which is incorporated herein by reference in its entirety.

### FIELD OF THE INVENTION

This invention relates to methods of preparing indolinone compounds, particularly pyrrole-substituted indolinone compounds having amide moieties on the pyrrole ring. The inventive methods are particularly useful in preparing indolinone compounds that are useful in the treatment of abnormal cell growth, such as cancer, in mammals.

### BACKGROUND

Classes of pyrrole-substituted indolinone compounds useful as therapeutic agents, particularly anti-cancer therapeutic agents, have previously been reported. Examples of such compounds and their synthesis can be found, for example, in U.S. Pat. Nos. 6,573,293 and 6,653,308; U.S. Patent Application Publication No. 2003/0229229, published Dec. 11, 2003; and U.S. Provisional Application No. 60/501,994, entitled “Method for Catalyzing Amidation Reactions,” filed Sep. 11, 2003, the disclosures of which are incorporated herein by reference in their entireties.

One method of synthesizing pyrrole-substituted indolinone compounds having amide moieties on the pyrrole ring, disclosed in the above-referenced U.S. Patent Application Publication No. 2003/0229229, proceeds via a pyrrole compound having aldehyde and acid moieties at the 5- and 3-positions, respectively, which is then coupled with an amine and an oxindole to form the desired pyrrole-substituted indolinone compound. This method, wherein the pyrrole ring is formed prior to attachment of the amide moiety, is an effective synthetic route. However, use of the acid-aldehyde substituted pyrrole compound results in consumption of excess amine due to formation of an imine-amide intermediate. It would be desirable to have alternative methods of synthesizing pyrrole-substituted indolinone compounds having amide substituents on the pyrrole ring that reduce or eliminate the need to use excess amine.

### SUMMARY

The invention provides methods of synthesizing pyrrole compounds, including pyrrole-substituted indolinone compounds having amide moieties on the pyrrole ring, wherein the pyrrole ring is formed having the desired amide group already in place. A method of synthesizing a specific compound, N-[2-(diethylamino)ethyl]-5-[(Z)-(5-fluoro-2-oxo-1,2-dihydro-3H-indol-3-ylidene)methyl]-2,4-dimethyl-1H-pyrrole-3-carboxamide, having formula 1a herein, 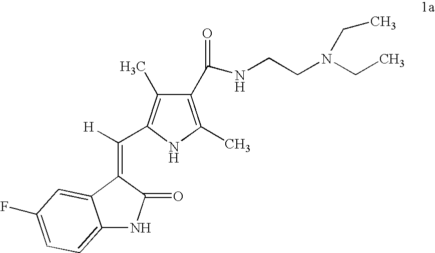
is disclosed in Manley, J. M.; Kalman, M. J.; Conway, B. G.; Ball, C. C.; Havens, J. L; and Vaidyanathan, R., “Early Amidation Approach to 3-(4-Amido)pyrrol-2-yl]-2-indolinones,” J. Org. Chem. 2003, 68, 6447-6450. Methods of synthesizing certain pyrrole compounds are disclosed in G.B. Patent No. 1,384,097. The disclosures of these documents are incorporated herein by reference in their entireties.

In one embodiment, the invention provides a method of preparing a compound of formula 1

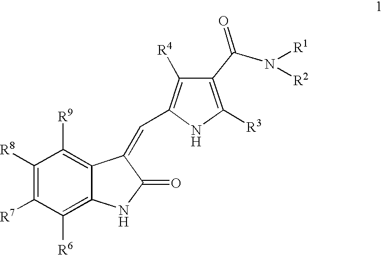

wherein:

R¹ is —(CH₂)ₘR¹⁰, and one or more hydrogens in the —(CH₂)ₘ groups is optionally substituted by —OH;

R² is H or C₁₋₁₂ alkyl;

optionally, R¹ and R², together with the nitrogen to which they are attached, can join to form a 5, 6 or 7-membered heterocyclic group optionally containing an additional N, O or S ring atom;

each R³ and R⁴ is independently C₁₋₁₂ alkyl;

each R⁶, R⁷, R⁸ and R⁹ is independently selected from the group consisting of hydrogen, C₁₋₁₂ alkyl, C₁₋₁₂ alkoxy, C₃₋₁₂ cycloalkyl, C₆₋₁₂ aryl, C₂₋₁₂ heterocyclic group containing 1 to 3 atoms selected from N, S and O, C₆₋₁₂ aryloxy, C₆₋₁₂ alkaryl, C₆₋₁₂ alkaryloxy, halogen, trihalomethyl, —S(O)R¹⁶, —SO₂NR¹⁶R¹⁷, —SO₃R¹⁶, —SR¹⁶, —NO₂, —NR¹⁶R¹⁷, —OH, —CN, —C(O)R¹⁶, —OC(O)R¹⁶, —NHC(O)R¹⁶, —(CH₂)ₙCO₂R¹⁶, and —CONR¹⁶R¹⁷;

R¹⁰ is selected from the group consisting of —NR¹¹R¹², —OH, —C(O)R¹³, C₆₋₁₂ aryl, C₆₋₁₂ alkaryl, C⁶⁻¹² aryloxy, C⁶⁻¹² alkaryloxy, C₁₋₁₂ alkoxy, —N⁺(O⁻)R¹¹R¹², —NHC(O)R¹⁴, and C₂₋₁₂ heterocyclic group containing 1 to 3 atoms selected from N, S and O;

R¹¹ and R¹² are independently selected from the group consisting of hydrogen, C₁₋₁₂ alkyl, C₁₋₁₂ cyanoalkyl, C₃₋₁₂ cycloalkyl, C₆₋₁₂ aryl, and C₂₋₁₂ heterocyclic group containing 1 to 3 atoms selected from N, S or O; or R¹¹ and R¹² may be combined to form a four-, five- or six-membered heterocyclic group optionally containing 1 to 3 atoms selected from N, O, or S in addition to the nitrogen atom to which R¹¹ and R¹² are bound, provided that the heterocyclic group formed by R¹¹ and R¹² may optionally be substituted by one or more R¹⁵ groups;

R¹³ is selected from the group consisting of —OH, C₁₋₁₂ alkyl, C₆₋₁₂ aryl, C₁₋₁₂ alkoxy, C₆₋₁₂ alkaryl, C₆₋₁₂ alkaryloxy and C₆₋₁₂ aryloxy;

R¹⁴ is selected from the group consisting of C₁₋₁₂ alkyl, C₁₋₁₂ haloalkyl, and C₆₋₁₂ aralkyl;

R¹⁵ is C₁₋₁₂ alkyl, C₁₋₁₂ cyanoalkyl, C₅₋₁₂ cycloalkyl, C₆₋₁₂ aryl or C₂₋₁₂ heterocyclic group containing 1 to 3 atoms selected from N, S and O;

R¹⁶ and R¹⁷ are independently selected from the group consisting of hydrogen, C₁₋₁₂ alkyl, C₁₋₁₂ cyanoalkyl, C₅₋₁₂ cycloalkyl, C₆₋₁₂ aryl, C₂₋₁₂ heterocyclic group containing 1 to 3 atoms selected from N, S and O, or in the group —NR¹⁶R¹⁷, R¹⁶ and R¹⁷ may be combined to form a four-, five- or six-membered heterocyclic group optionally containing 1 to 3 atoms selected from N, O, or S in addition to the nitrogen atom to which R¹⁶ and R¹⁷ are bound;

m is 0, 1, 2, 3 or 4; and

n is 0, 1 or 2;
the method comprising reacting a compound of formula 15 with a compound of formula 17 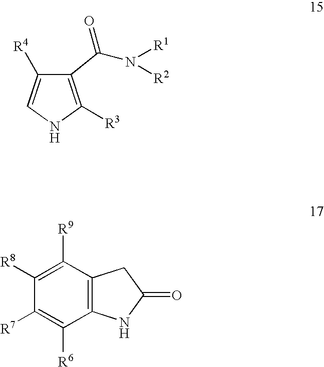
and a formylating reagent to form the compound of formula 1.

In a particular aspect of this embodiment, R¹⁰ is —NR¹¹R¹² and R¹¹ and R¹² are independently H or C₁₋₄ alkyl.

In another particular aspect of this embodiment, R¹⁰ is —NR¹¹R¹², where R¹¹ and R¹², together with the nitrogen atom to which they are bound, are combined to form a five or six-membered heterocyclic group optionally containing an additional N, O or S ring atom.

In another particular aspect of this embodiment, and in combination with any other particular aspect not inconsistent, m is 2, 3 or 4 and R¹⁰ is a heterocyclic group selected from 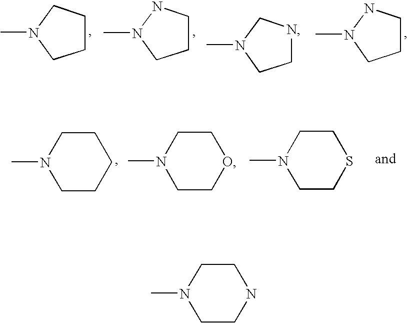
optionally substituted by one or more R¹⁵ groups.

In another particular aspect of this embodiment, and in combination with any other particular aspect not inconsistent, the compound of formula 1 is selected from the group consisting of

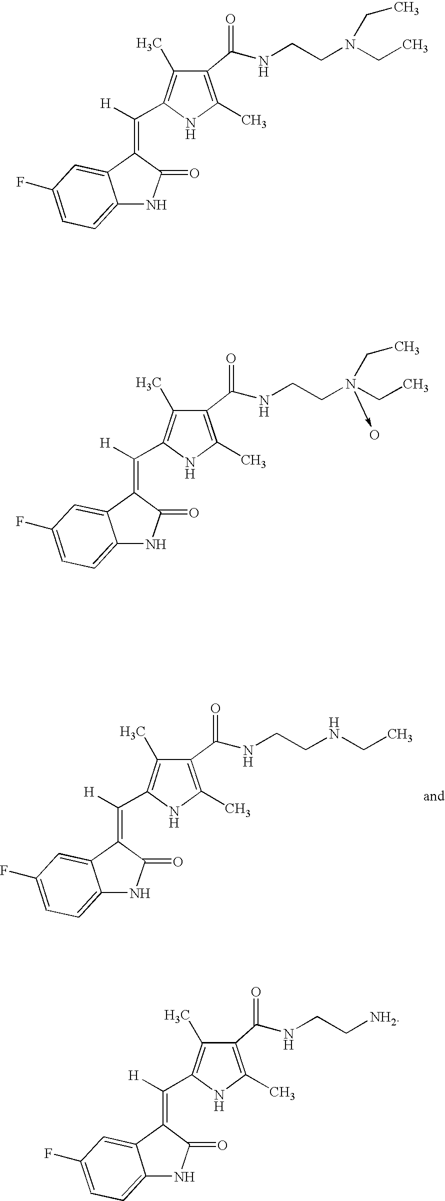

In another particular aspect of this embodiment, and in combination with any other particular aspect not inconsistent, the compound of formula 1 is selected from the group consisting of:

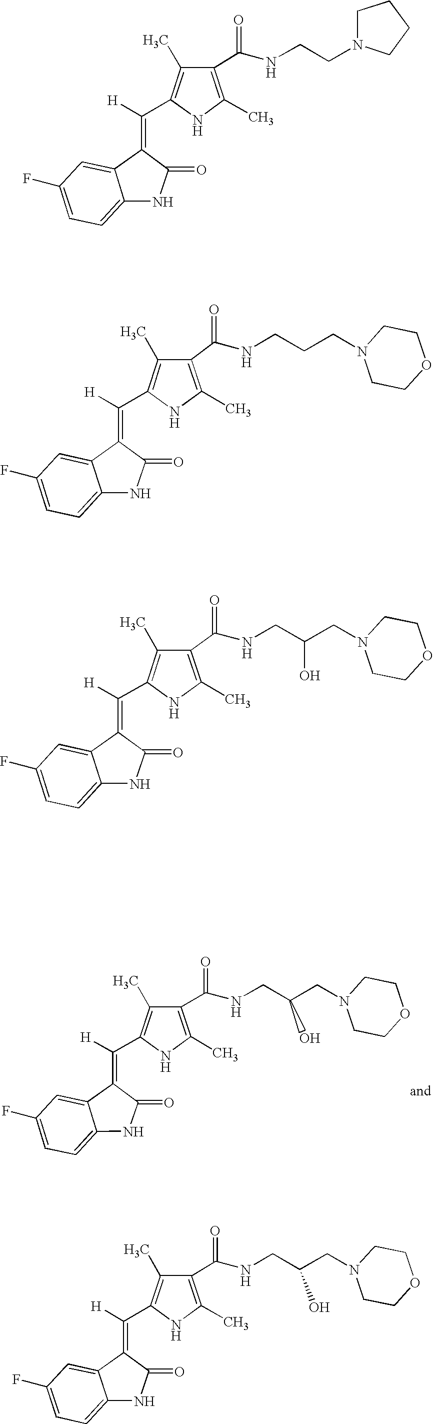

In another particular aspect of this embodiment, and in combination with any other particular aspect not inconsistent, the formylating agent is a halo-substituted iminium salt, such as, for example, chloromethylenedimethylammonium chloride.

In another particular aspect of this embodiment, and in combination with any other particular aspect not inconsistent, the formylating agent is formed in situ from DMF in POCl₃.

In another particular aspect of this embodiment, and in combination with any other particular aspect not inconsistent, the formylating agent is a trialkylorthoformate.

In another particular aspect of this embodiment, and in combination with any other particular aspect not inconsistent, the step of reacting the compound of formula 15 with the compound of formula 17 and the formylating agent is carried out by (i) reacting the compound of formula 15 with the formylating agent to form an intermediate; and (ii) reacting the intermediate with the compound of formula 17 to form the compound of formula 1. In a specific aspect, the formylating agent is a compound of formula A-L, where L is a halogen or a leaving group, A is a formyl group or a group that can be hydrolyzed to a formyl group, and the intermediate is a compound of formula 16 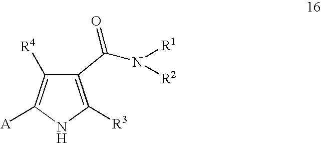
In a further specific aspect, the compound of formula A-L is a halo-substituted iminium salt such as, for example, chloromethylenedimethylammonium chloride, and the intermediate is a compound of formula:

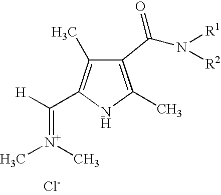

In another particular aspect of this embodiment, and in combination with any other particular aspect not inconsistent, the compound of formula A-L is a trialkylorthoformate, and the intermediate is a compound of formula: 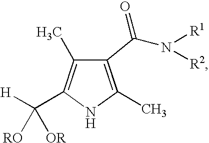
wherein each R group is independently C₁₋₁₂ alkyl.

In another particular aspect of this embodiment, and in combination with any other particular aspect not inconsistent, the compound of formula 15 is formed by decarboxylating a compound of formula 14 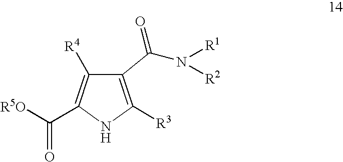
wherein R₅ is a C₁₋₁₂ alkyl group to form the compound of formula 15. In a further aspect of this embodiment, the compound of formula 14 is formed by reacting a compound of formula 12 with a compound of formula 13 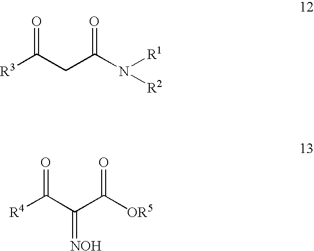
under pyrrole formation conditions. Particular pyrrole formation conditions include, for example, carrying out the reaction in the presence of zinc and acetic acid, or carrying out the reaction in the presence of H₂ and a hydrogenation catalyst.

In another embodiment, the invention provides a method of preparing a pyrrole of formula 14

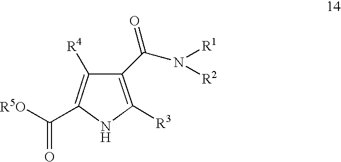

wherein:

R¹ is —(CH₂)ₘR¹⁰, and one or more hydrogens in the —(CH₂)ₘ groups is optionally substituted by —OH;

R² is H or C₁₋₁₂ alkyl;

optionally, R¹ and R², together with the nitrogen to which they are attached, can join to form a 5, 6 or 7-membered heterocyclic group optionally containing an additional N, O or S ring atom;

each R³ and R⁴ is independently C₁₋₁₂ alkyl;

R₅ is C₁₋₁₂ alkyl;

R¹⁰ is selected from the group consisting of —NR¹¹R¹², —OH, —C(O)R¹³, C₆₋₁₂ aryl, C₆₋₁₂ alkaryl, C₆₋₁₂ aryloxy, C₆₋₁₂ alkaryloxy, C₁₋₁₂ alkoxy, —N⁺(O⁻)R¹¹R¹², —NHC(O)R¹⁴, and C₂₋₁₂ heterocyclic group containing 1 to 3 atoms selected from N, S and O;

R¹¹ and R¹² are independently selected from the group consisting of hydrogen, C₁₋₁₂ alkyl, C₁₋₁₂ cyanoalkyl, C₃₋₁₂ cycloalkyl, C₆₋₁₂ aryl, and C₂₋₁₂ heterocyclic group containing 1 to 3 atoms selected from N, S or O; or R¹¹ and R¹² may be combined to form a four-, five- or six-membered heterocyclic group optionally containing 1 to 3 atoms selected from N, O, or S in addition to the nitrogen atom to which R¹¹ and R¹² are bound, provided that the heterocyclic group formed by R¹¹ and R¹² may optionally be substituted by one or more R¹⁵ groups;

R¹³ is selected from the group consisting of —OH, C₁₋₁₂ alkyl, C₆₋₁₂ aryl, C₁₋₁₂ alkoxy, C₆₋₁₂ alkaryl, C₆₋₁₂ alkaryloxy and C₆₋₁₂ aryloxy;

R¹⁴ is selected from the group consisting of C₁₋₁₂ alkyl, C₁₋₁₂ haloalkyl, and C₆₋₁₂ aralkyl;

R¹⁵ is C₁₋₁₂ alkyl, C₁₋₁₂ cyanoalkyl, C₅₋₁₂ cycloalkyl, C₆₋₁₂ aryl or C₂₋₁₂ heterocyclic group containing 1 to 3 atoms selected from N, S and O; and

m is 0, 1, 2, 3 or 4;
the method comprising reacting a compound of formula 12 with a compound of formula 13 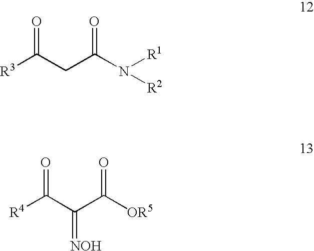
under pyrrole formation conditions to form the compound of formula 14.

In a particular aspect of this embodiment, R¹⁰ is —NR¹¹R¹² and R¹¹ and R¹² are independently H or C₁₋₄ alkyl.

In another particular aspect of this embodiment, R¹⁰ is —NR¹¹R¹², where R¹¹ and R¹², together with the nitrogen atom to which they are bound, are combined to form a five or six-membered heterocyclic group optionally containing an additional N, O or S ring atom.

In another particular aspect of this embodiment, and in combination with any other particular aspect not inconsistent, m is 2, 3 or 4 and R¹⁰ is a heterocyclic group selected from 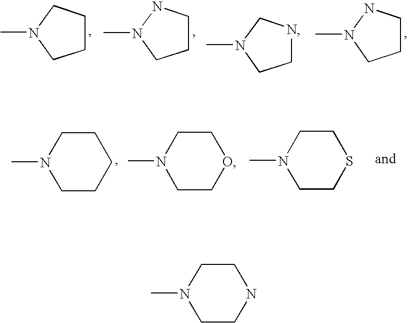
optionally substituted by one or more R¹⁵ groups.

In another particular aspect of this embodiment, and in combination with any other particular aspect not inconsistent, the compound of formula 14 is selected from the group consisting of

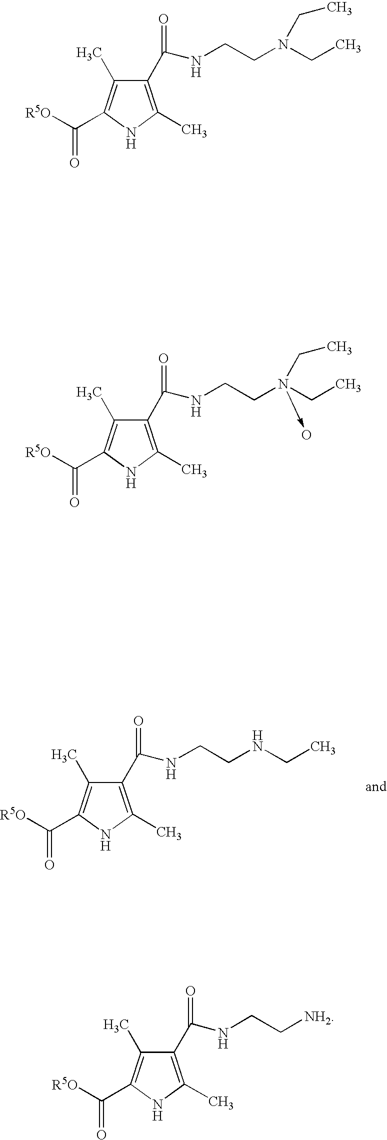

In another particular aspect of this embodiment, and in combination with any other particular aspect not inconsistent, the compound of formula 1 is selected from the group consisting of:

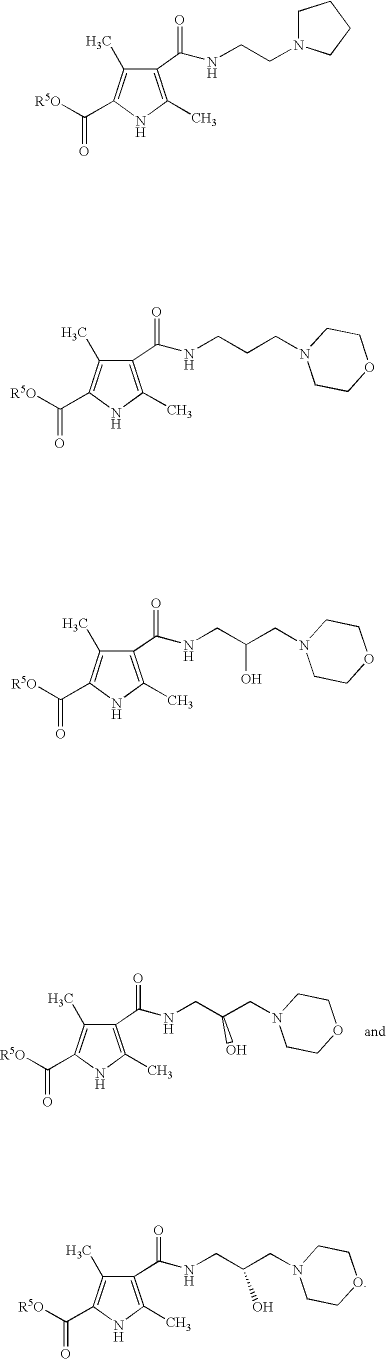

In another particular aspect of this embodiment, and in combination with any other particular aspect not inconsistent, the pyrrole formation conditions comprises carrying out the reaction in the presence of zinc and acetic acid.

In another particular aspect of this embodiment, and in combination with any other particular aspect not inconsistent, the pyrrole formation conditions comprises carrying out the reaction in the presence of H₂ and a hydrogenation catalyst.

In a further aspect of this embodiment, the method further comprises decarboxylating the compound of formula 14 to form a compound of formula 15 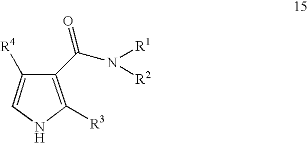
In still a further aspect, the method further comprises reacting the compound of formula 15 with a compound of formula 17 and a formylating agent to form a compound of formula 1

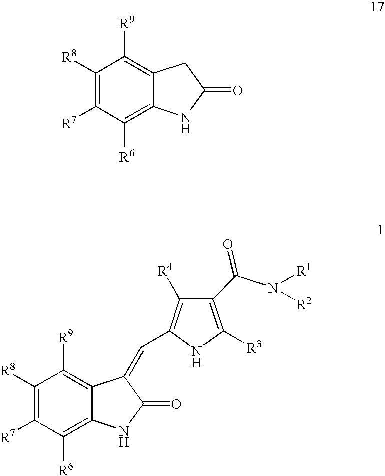

wherein

each R⁶, R⁷, R⁸ and R⁹ is independently selected from the group consisting of hydrogen, C₁₋₁₂ alkyl, C₁₋₁₂ alkoxy, C₃₋₁₂ cycloalkyl, C₆₋₁₂ aryl, C₂₋₁₂ heterocyclic group containing 1 to 3 atoms selected from N, S and O, C₆₋₁₂ aryloxy, C₁₋₁₂ alkaryl, C₆₋₁₂ alkaryloxy, halogen, trihalomethyl, —S(O)R¹⁶, —SO₂NR¹⁶R¹⁷, —SO₃R¹⁶, —SR¹⁶, —NO₂, —NR¹⁶R¹⁷, —OH, —CN, —C(O)R¹⁶, —OC(O)R¹⁶, —NHC(O)R¹⁶, —(CH₂)ₙCO₂R¹⁶, and —CONR¹⁶R¹⁷;

R¹⁶ and R¹⁷ are independently selected from the group consisting of hydrogen, C₁₋₁₂ alkyl, C₁₋₁₂ cyanoalkyl, C₅₋₁₂ cycloalkyl, C₆₋₁₂ aryl, C₂₋₁₂ heterocyclic group containing 1 to 3 atoms selected from N, S and O, or in the group —NR¹⁶R¹⁷, R¹⁶ and R¹⁷ may be combined to form a four-, five- or six-membered heterocyclic group optionally containing 1 to 3 atoms selected from N, O, or S in addition to the nitrogen atom to which R¹⁶ and R¹⁷ are bound; and

n is 0, 1 or 2.

In this aspect, examples of suitable formylating agents include: halo-substituted iminium salts, such as chloromethylenedimethylammonium chloride; in-situ formed iminium compounds, such as from DMF and POCl₃; and trialkylorthoformates, such as trimethylorthoformate (TMOF).

In another particular aspect of this embodiment, and in combination with any other particular aspect not inconsistent, the step of reacting the compound of formula 15 with the compound of formula 17 and the formylating agent is carried out by (i) reacting the compound of formula 15 with the formylating agent to form an intermediate; and (ii) reacting the intermediate with the compound of formula 17 to form the compound of formula 1. In a specific aspect, the formylating agent is a compound of formula A-L, where L is a halogen or a leaving group, A is a formyl group or a group that can be hydrolyzed to a formyl group, and the intermediate is a compound of formula 16 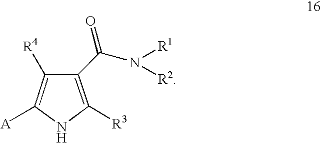
In this aspect, examples of compounds of formula AL include halo-substituted iminium salts, such as chloromethylenedimethylammonium chloride, and the intermediate is a compound of formula: 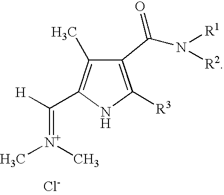
or trialkylorthoformates, such as trimethylorthoformate, and the intermediate is a compound of formula: 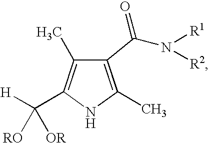
wherein each R group is independently C₁₋₁₂ alkyl.
In this aspect, in a specific example, the compound of formula 1 is selected from the group consisting of 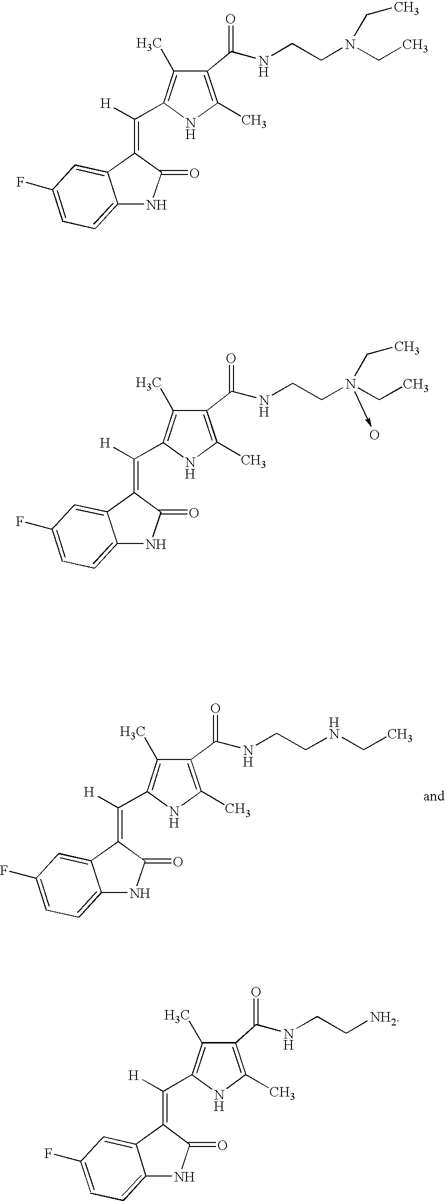
In another specific example, the compound of formula 1 is selected from the group consisting of:

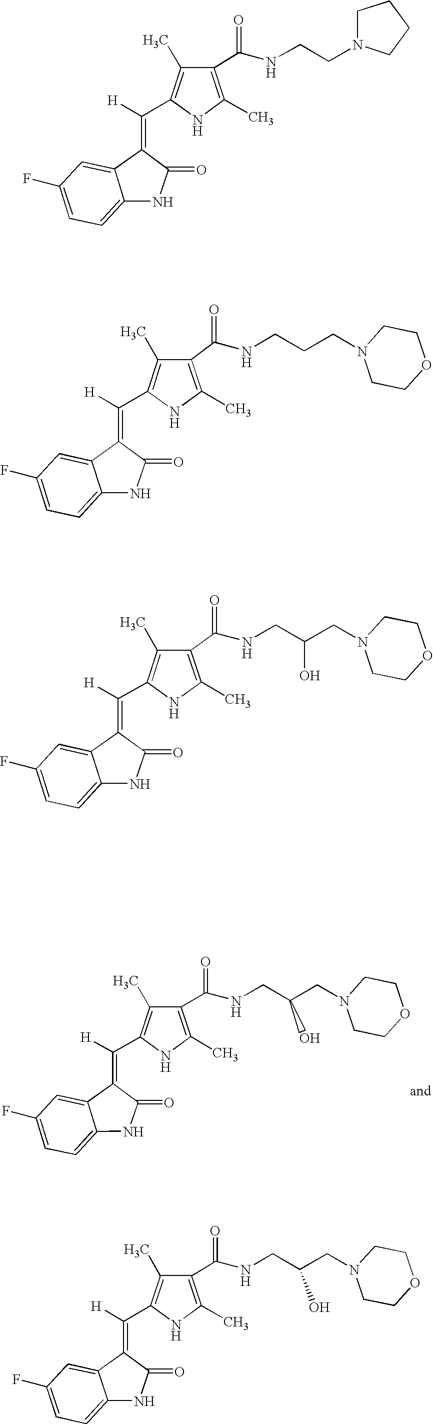

In any of the inventive methods herein, the method optionally further comprises forming a salt, preferably a pharmaceutically acceptable salt, of the compounds, particularly compounds of formula 1. Examples of suitable salts of compounds of formula 1 can be found, for example, in U.S. Patent Application Publication No. 2003/0069298, the disclosure of which is incorporated herein by reference in its entirety.

Examples of the use of the indolinone compounds described herein can be found in, for example, U.S. Pat. Nos. 6,573,293 and 6,653,308; U.S. Patent Application Publication Nos. 2003/0216410 and 2003/0130280; and PCT Publication Nos. WO 2004/024127 and 2004/045523, the disclosures of which are incorporated herein by reference in their entireties.

Definitions

Unless otherwise stated, the following terms used in the specification and claims have the meanings discussed below.

“Alkyl” refers to a saturated aliphatic hydrocarbon radical including straight chain and branched chain groups of 1 to 20 carbon atoms, preferably 1 to 12 carbon atoms, more preferably 1 to 8 carbon atoms, or 1 to 6 carbon atoms, or 1 to 4 carbon atoms. “Lower alkyl” refers specifically to an alkyl group with 1 to 4 carbon atoms. Examples of alkyl groups include methyl, ethyl, propyl, 2-propyl, n-butyl, iso-butyl, tert-butyl, pentyl, and the like.

“Cycloalkyl” refers to a 3 to 8 member all-carbon monocyclic ring, an all-carbon 5-member/6-member or 6-member/6-member fused bicyclic ring, or a multicyclic ring which may or may not be fused (a “fused” ring system means that each ring in the system shares an adjacent pair of carbon atoms with each other ring in the system), wherein one or more of the rings may contain one or more double bonds but none of the rings has a completely conjugated pi-electron system. Examples, without limitation, of cycloalkyl groups are cyclopropane, cyclobutane, cyclopentane, cyclopentene, cyclohexane, cyclohexadiene, adamantane, cycloheptane, cycloheptatriene, and the like. Illustrative examples of cycloalkyl groups are derived from, but not limited to, the following:

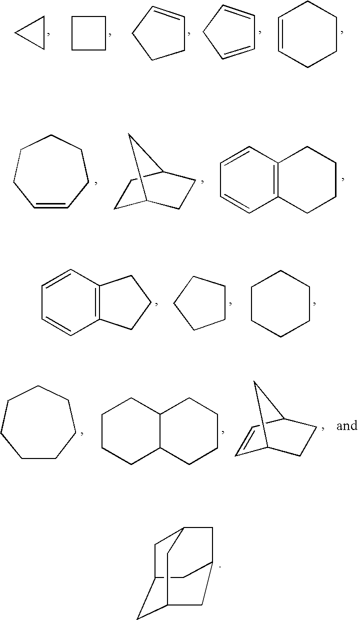

“Alkenyl” refers to an alkyl group, as defined herein, consisting of at least two carbon atoms and at least one carbon-carbon double bond. Representative examples include, but are not limited to, ethenyl, 1-propenyl, 2-propenyl, 1-, 2-, or 3-butenyl, and the like.

“Alkynyl” refers to an alkyl group, as defined herein, consisting of at least two carbon atoms and at least one carbon-carbon triple bond. Representative examples include, but are not limited to, ethynyl, 1-propynyl, 2-propynyl, 1-, 2-, or 3-butynyl, and the like.

“Aryl” refers to an all-carbon monocyclic or fused-ring polycyclic groups of 6 to 12 carbon atoms having a completely conjugated pi-electron system. Examples, without limitation, of aryl groups are phenyl, naphthalenyl and anthracenyl.

“Heteroaryl” refers to a monocyclic or fused ring group of 5 to 12 ring atoms containing one, two, three or four ring heteroatoms selected from N, O, and S, the remaining ring atoms being C, and, in addition, having a completely conjugated pi-electron system. Examples, without limitation, of heteroaryl groups are pyrrole, furan, thiophene, imidazole, oxazole, thiazole, pyrazole, pyridine, pyrimidine, quinoline, isoquinoline, purine, tetrazole, triazine, and carbazole.

Examples of typical monocyclic heteroaryl groups include, but are not limited to:

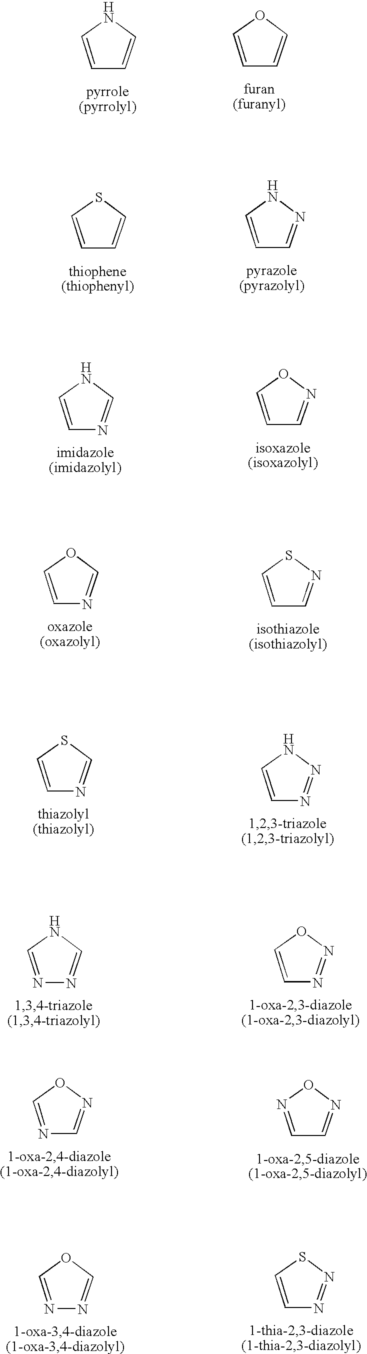

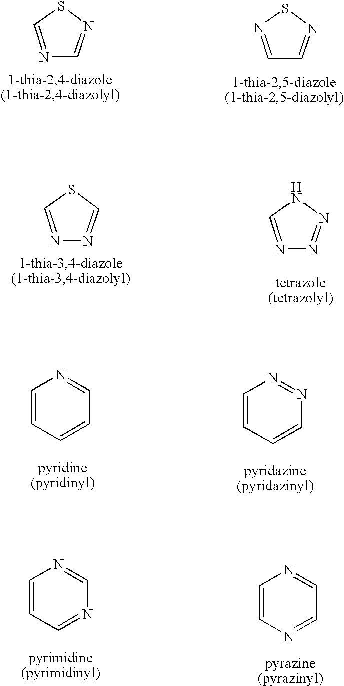

Examples of suitable fused ring heteroaryl groups include, but are not limited to:

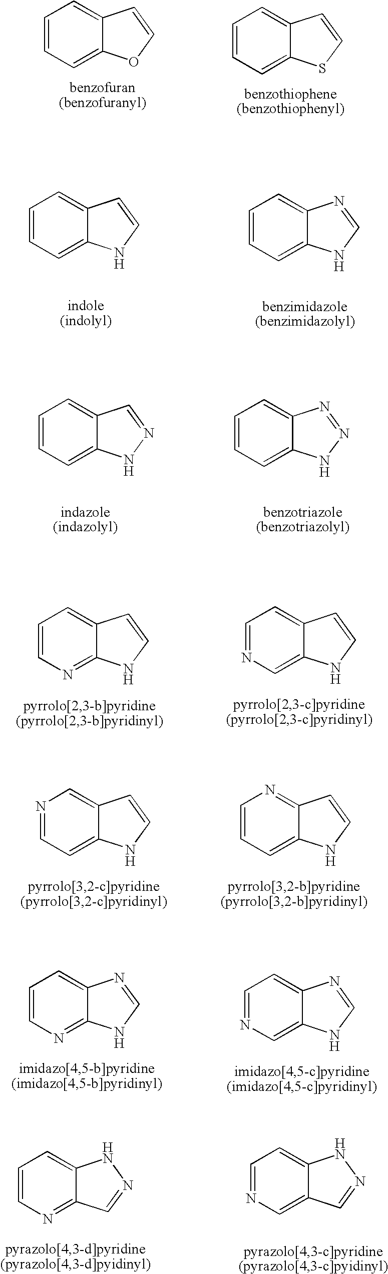

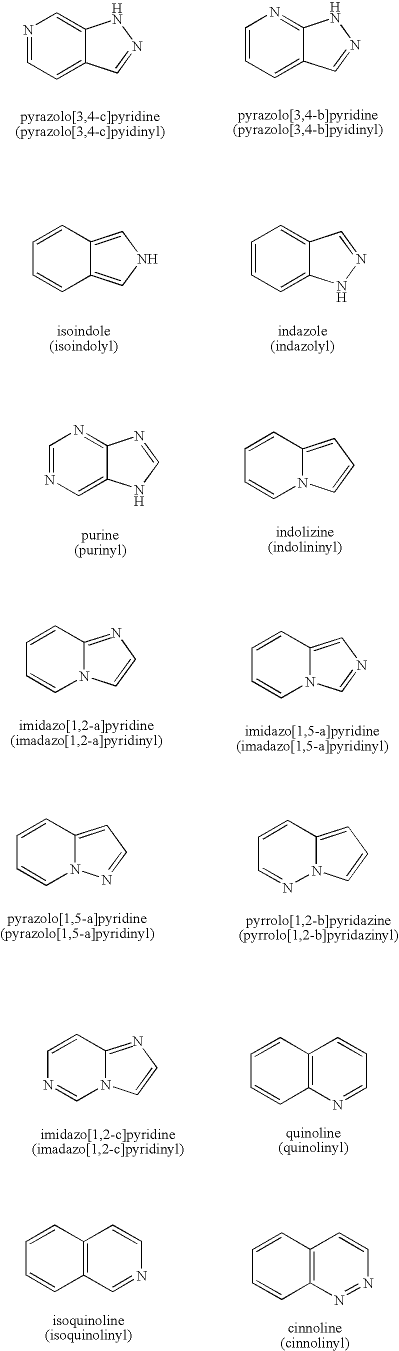

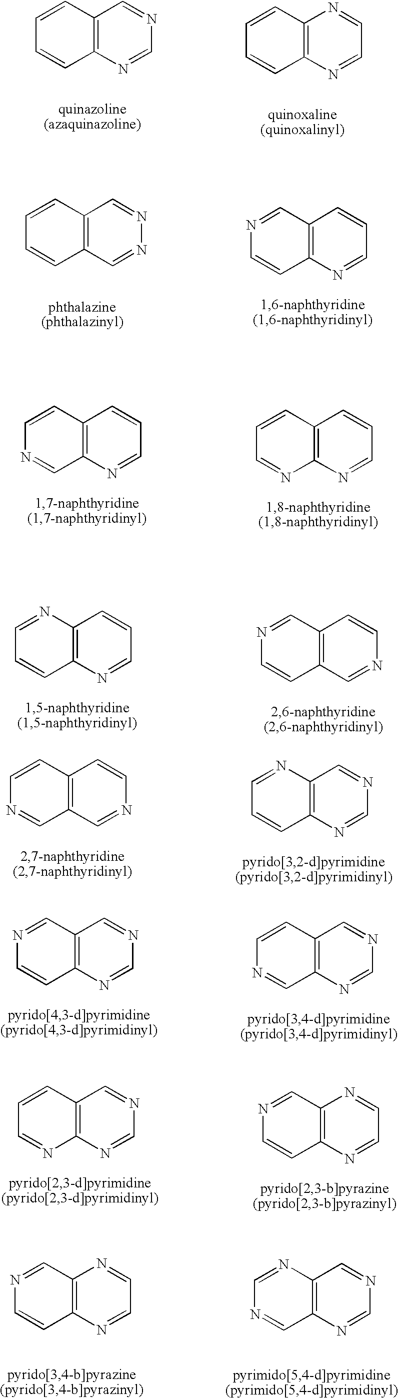

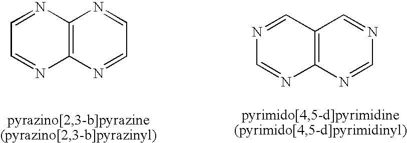

“Heteroalicyclic” or “heterocycle” refers to a monocyclic or fused ring group having in the ring(s) of 3 to 12 ring atoms, in which one or two ring atoms are heteroatoms selected from N, O, and S(O)ₙ (where n is 0, 1 or 2), the remaining ring atoms being C. The rings may also have one or more double bonds. However, the rings do not have a completely conjugated pi-electron system. Examples of suitable saturated heteroalicyclic groups include, but are not limited to:

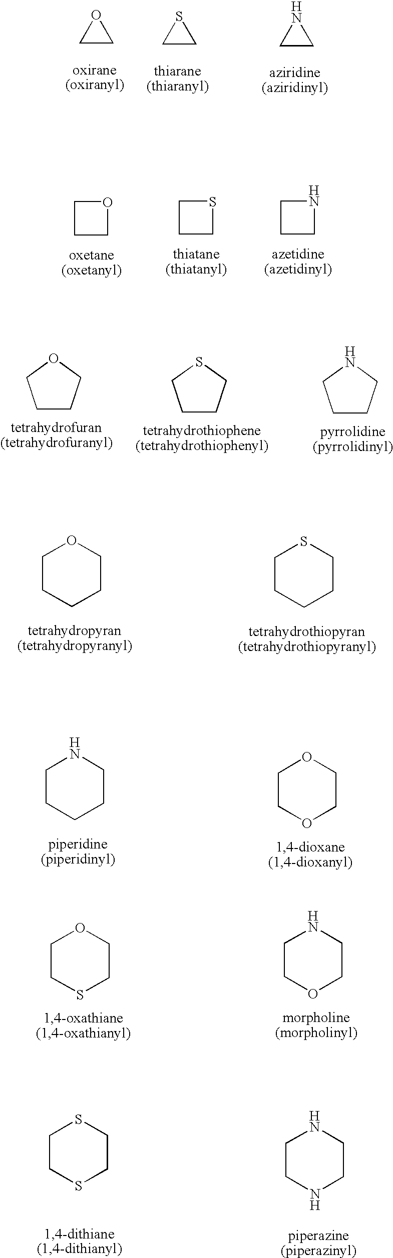

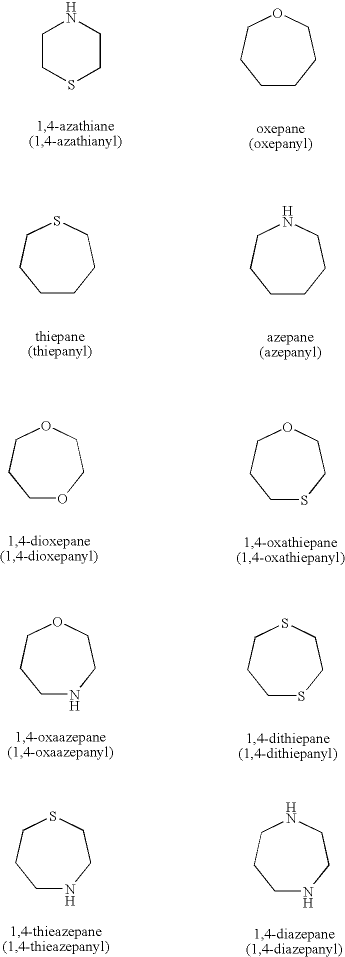

Examples of suitable partially unsaturated heteroalicyclic groups include, but are not limited to:

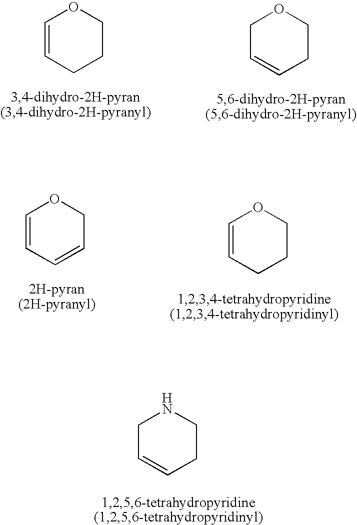

“Hydroxy” refers to an —OH group.

“Alkoxy” refers to both an —O-(alkyl) or an —O-(unsubstituted cycloalkyl) group. Representative examples include, but are not limited to, methoxy, ethoxy, propoxy, butoxy, cyclopropyloxy, cyclobutyloxy, cyclopentyloxy, cyclohexyloxy, and the like.

“Haloalkoxy” refers to an —O-(haloalkyl) group. Representative examples include, but are not limited to, trifluoromethoxy, tribromomethoxy, and the like.

“Aryloxy” refers to an —O-aryl or an —O-heteroaryl group, as defined herein. Representative examples include, but are not limited to, phenoxy, pyridinyloxy, furanyloxy, thienyloxy, pyrimidinyloxy, pyrazinyloxy, and the like, and derivatives thereof.

“Mercapto” refers to an —SH group.

“Alkylthio” refers to an —S-(alkyl) or an —S-(unsubstituted cycloalkyl) group. Representative examples include, but are not limited to, methylthio, ethylthio, propylthio, butylthio, cyclopropylthio, cyclobutylthio, cyclopentylthio, cyclohexylthio, and the like.

“Arylthio” refers to an —S-aryl or an —S-heteroaryl group, as defined herein. Representative examples include, but are not limited to, phenylthio, pyridinylthio, furanylthio, thienylthio, pyrimidinylthio, and the like and derivatives thereof.

“Acyl” or “carbonyl” refers to a —C(O)R″ group, where R″ is selected from the group consisting of hydrogen, lower alkyl, trihalomethyl, cycloalkyl, aryl, heteroaryl (bonded through a ring carbon) and heteroalicyclic (bonded through a ring carbon). Representative acyl groups include, but are not limited to, acetyl, trifluoroacetyl, benzoyl, and the like

“Aldehyde” refers to an acyl group in which R″ is hydrogen.

“Thioacyl” or “thiocarbonyl” refers to a —C(S)R″ group, with R″ as defined above.

A “thiocarbonyl” group refers to a —C(S)R″ group, with R″ as defined above.

A “C-carboxy” group refers to a —C(O)OR″ group, with R″ as defined above.

An “O-carboxy” group refers to a —OC(O)R″ group, with R″ as defined above.

“Ester” refers to a —C(O)OR″ group with R″ as defined herein except that R″ cannot be hydrogen.

“Acetyl” group refers to a —C(O)CH₃ group.

“Halo” group refers to fluorine, chlorine, bromine or iodine, preferably fluorine or chlorine.

“Trihalomethyl” group refers to a methyl group having three halo substituents, such as a trifluoromethyl group.

“Cyano” refers to a —C≡N group.

A “sulfinyl” group refers to a —S(O)R″ group wherein, in addition to being as defined above, R″ may also be a hydroxy group.

A “sulfonyl” group refers to a —S(O)₂R″ group wherein, in addition to being as defined above, R″ may also be a hydroxy group.

“S-sulfonamido” refers to a —S(O)₂NR^{x}R^{y} group, with R^{x} and R^{y} as defined above.

“N-sulfonamido” refers to a —NR^{x}S(O)₂R^{y} group, with R^{x} and R^{y} as defined above.

“O-carbamyl” group refers to a —OC(O)NR^{x}R^{y} group with R^{x} and R^{y} as defined above.

“N-carbamyl” refers to an R^{y}OC(O)NR^{x}— group, with R^{x} and R^{y} as defined above.

“O-thiocarbamyl” refers to a —OC(S)NR^{x}R^{y} group with R^{x} and R^{y} as defined above.

“N-thiocarbamyl” refers to a R^{y}OC(S)NR^{x}— group, with R^{y} and R^{x} as defined above.

“Amino” refers to an —NR^{x}R^{y} group, wherein R^{x} and R^{y} are both hydrogen.

“C-amido” refers to a —C(O)NR^{x}R^{y} group with R^{x} and R^{y} as defined above.

“N-amido” refers to a R^{x}C(O)NR^{y}— group, with R^{x} and R^{y} as defined above.

“Nitro” refers to a —NO₂ group.

“Haloalkyl” means an alkyl, preferably lower alkyl, that is substituted with one or more same or different halo atoms, e.g., —CH₂Cl, —CF₃, —CH₂CF₃, —CH₂CCl₃, and the like.

“Hydroxyalkyl” means an alkyl, preferably lower alkyl, that is substituted with one, two, or three hydroxy groups; e.g., hydroxymethyl, 1 or 2-hydroxyethyl, 1,2-, 1,3-, or 2,3-dihydroxypropyl, and the like.

“Aralkyl” means alkyl, preferably lower alkyl, that is substituted with an aryl group as defined above; e.g., —CH₂phenyl, —(CH₂)₂phenyl, —(CH₂)₃phenyl, CH₃CH(CH₃)CH₂phenyl,and the like and derivatives thereof.

“Heteroaralkyl” group means alkyl, preferably lower alkyl, that is substituted with a heteroaryl group; e.g., —CH₂pyridinyl, —(CH₂)₂pyrimidinyl, —(CH₂)₃imidazolyl, and the like, and derivatives thereof.

“Monoalkylamino” means a radical —NHR where R is an alkyl or unsubstituted cycloalkyl group; e.g., methylamino, (1-methylethyl)amino, cyclohexylamino, and the like.

“Dialkylamino” means a radical —NRR where each R is independently an alkyl or unsubstituted cycloalkyl group; dimethylamino, diethylamino, (1-methylethyl)-ethylamino, cyclohexylmethylamino, cyclopentylmethylamino, and the like.

“Optional” or “optionally” means that the subsequently described event or circumstance may but need not occur, and that the description includes instances where the event or circumstance occurs and instances in which it does not. For example, “heterocycle group optionally substituted with an alkyl group” means that the alkyl may but need not be present, and the description includes situations where the heterocycle group is substituted with an alkyl group and situations where the heterocycle group is not substituted with the alkyl group.

A “pharmaceutical composition” refers to a mixture of one or more of the compounds described herein, or physiologically/pharmaceutically acceptable salts, solvates, hydrates or prodrugs thereof, with other chemical components, such as physiologically/pharmaceutically acceptable carriers and excipients. The purpose of a pharmaceutical composition is to facilitate administration of a compound to an organism.

As used herein, a “physiologically/pharmaceutically acceptable carrier” refers to a carrier or diluent that does not cause significant irritation to an organism and does not abrogate the biological activity and properties of the administered compound.

A “pharmaceutically acceptable excipient” refers to an inert substance added to a pharmaceutical composition to further facilitate administration of a compound. Examples, without limitation, of excipients include calcium carbonate, calcium phosphate, various sugars and types of starch, cellulose derivatives, gelatin, vegetable oils and polyethylene glycols.

As used herein, the term “pharmaceutically acceptable salt” refers to those salts which retain the biological effectiveness and properties of the parent compound. Such salts include:

(i) acid addition salts, which can be obtained by reaction of the free base of the parent compound with inorganic acids such as hydrochloric acid, hydrobromic acid, nitric acid, phosphoric acid, sulfuric acid, and perchloric acid and the like, or with organic acids such as acetic acid, oxalic acid, (D) or (L) malic acid, maleic acid, methanesulfonic acid, ethanesulfonic acid, ptoluenesulfonic acid, salicylic acid, tartaric acid, citric acid, succinic acid or malonic acid and the like; or

(2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like.

### DETAILED DESCRIPTION

Scheme I illustrates a general reaction scheme for carrying out methods of the invention.

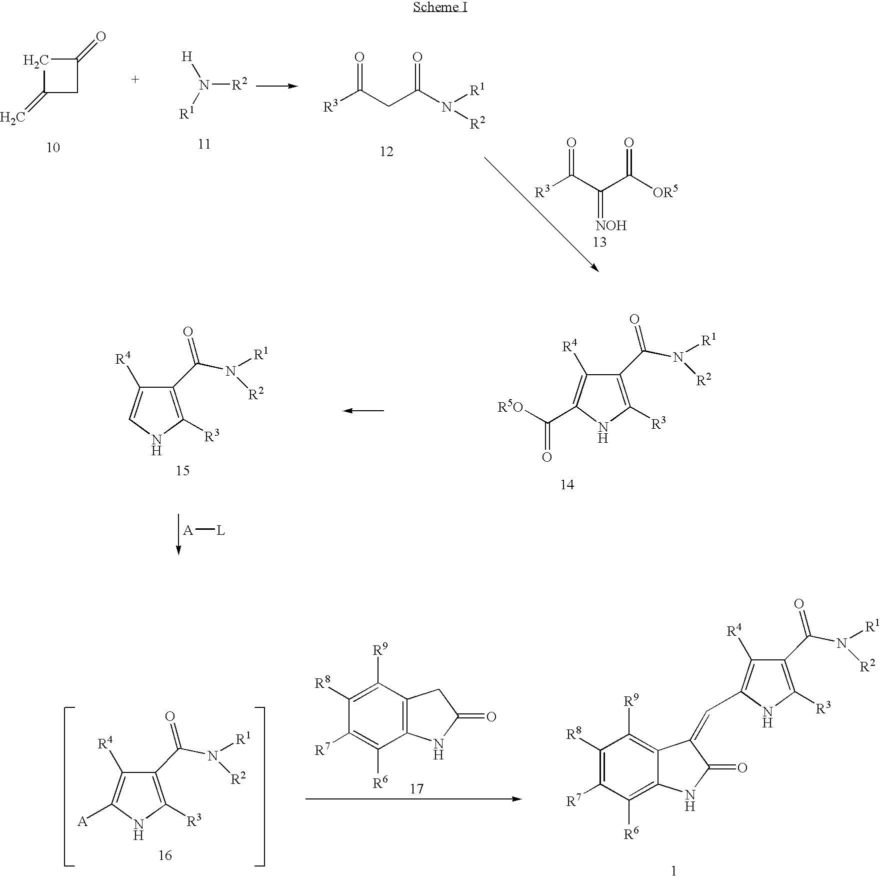

The β-ketoamide 12 is readily formed by reaction of diketene 10 with the desired amine 11, in an organic solvent such as methyl t-butyl ether (MTBE), THF, benzene, etc. The reaction can take place at any convenient temperature, typically room temperature. Certain β-ketoamides, such as 12a described below, are prone to decomposition, and are best used immediately, or stored at low temperatures, such as about −20° C.

Oxime 13 is conveniently obtained by reaction of the corresponding R⁵ acetoacetate ester with a nitrite salt, such as sodium nitrite, under acidic conditions, such as in acetic acid. The oxime 13 is then reacted with β-ketoamide 12 under pyrrole formation conditions to form pyrrole 14. Suitable pyrrole formation conditions include the well-known Knorr formation conditions, wherein the oxime and amide are reacted in the presence of zinc and acetic acid to form the pyrrole. Alternatively, pyrrole formation can be effected by hydrogenating a mixture of amide 12 and oxime 13 over a suitable catalyst, such as 10 wt % Pd/C in acetic acid. Typical hydrogenation conditions are 45 psig at a temperature of 60-70° C., for a period of about 1 to 7 hours, preferably with a dry catalyst.

Pyrrole 14 is then decarboxylated to form the alpha free pyrrole 15. Decarboxylation reactions are well known, and one skilled in the art can readily determine suitable decarboxylation conditions for a particular pyrrole 14. For the specific case of pyrrole 14a described below, it was found that decarboxylation using HCl/EtOH formed the desired product 15a, but also formed a dimer of the pyrrole. Use of 1 M H₂SO₄ in MeOH (3:1 in H₂O) at 65° C. led to clean formation of the product 15a without any trace of the dimer. Similarly, the use of trifluoroacetic acid at room temperature also cleanly produced the desired alpha free pyrrole.

The alpha free pyrrole 15 is then reacted with oxindole 17 and a formylating agent to form the compound of formula 1. Suitable formylating agents are those capable of reacting with pyrrole 15 to provide a group A at the 2-position of the pyrrole ring, where A is a —CH(O), —CH(OR)₂ or —CH(NR′R″) group, and R, R′ and R″ are independently C₁₋₁₂ alkyl. The formylation reaction is shown schematically in Scheme 1 as producing intermediate 16, but this intermediate need not be, and is not typically, isolated. In Scheme 1, group A is provided via a reagent AL, where L is a halogen or a leaving group. Examples of formylating agents include Vilsmeier reagents, typically iminium salts. A specific formylating agent is chloromethylenedimethylammonium chloride. The reaction of pyrrole 15 with the formylating agent and oxindole 17 can proceed sequentially or simultaneously, as desired. Other examples of formylating agents include in-situ-formed iminium compounds, such as from DMF in POCl₃.

As an alternative to the sequential decarboxylation and formylation reactions shown in scheme 1, decarboxylation and formylation can be carried out in a single pot reaction. Thus, pyrrole 14 can be reacted with trifluoroacetic acid (TFA), preferably at a temperature below room temperature to minimize dimer formation, and a trialkylorthoformate, HC(OR₃), where each R is independently C₁₋₁₂ alkyl, preferably C₁₋₄ alkyl, more preferably methyl. The aldehyde product 24 is then reacted with oxindole 17 to form the product 1.

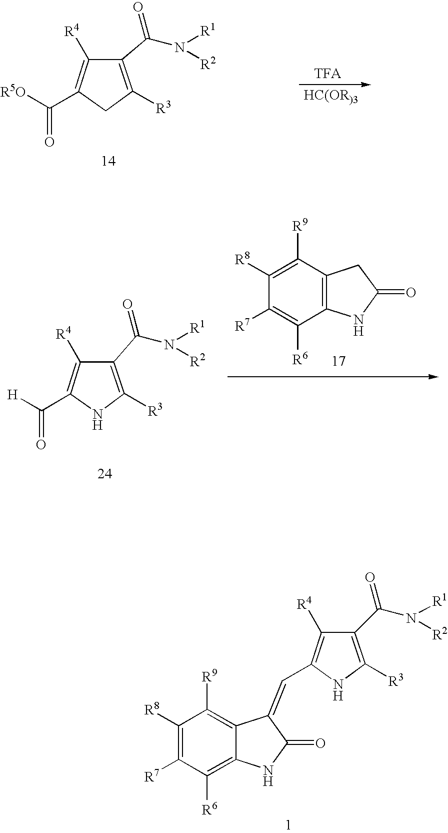

If desired, the product 1 can be further reacted with a suitable acid to form a salt, preferably a pharmaceutically acceptable salt. Alternatively, the salt can be formed simultaneously with the formylation/oxindole reaction, as shown in Example 2 herein.

Examples of specific syntheses are shown in the Examples herein.

### EXAMPLES

As used herein, “Et” means ethyl, “Ac” means acetyl, “Me” means methyl, “MeOH” means methanol, “TBME” or “MTBE” means t-butyl methyl ether, “TLC” means thin layer chromatography, Unless otherwise indicated, compounds described herein can be obtained from commercial sources, or prepared using procedures known in the literature.

### Example 1

### Preparation of N-[2-(diethylamino)ethyl]-5-[(Z)-(5-fluoro-2-oxo-1,2-dihydro-3H-indol-3-ylidene)methyl]-2,4-dimethyl-1H-pyrrole-3-carboxamide (1a)

Preparation of the compound of formula 1a, and compounds used in the synthesis, was as follows.

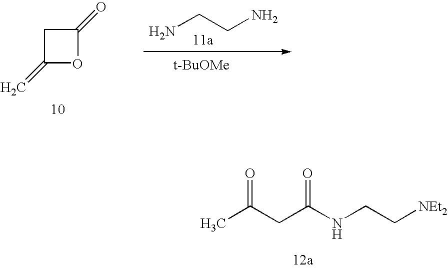

N-[2-(diethylamino)ethyl]-3-oxobutanamide (12a). Diketene (10) (30.0 g; 357 mmol) was added to a 1000 mL 3-neck round-bottomed flask equipped with an addition funnel, N₂ inlet, and overhead stirrer. tert-Butyl methyl ether (500 mL) was transferred to the flask and the solution was cooled to 0-5° C. using an ice-water bath. N,N-Diethylethylenediamine (11a) (33.3 g; 287 mmol) was added to the solution drop-wise, maintaining the temperature below 5° C. The ice-water bath was removed and the solution was allowed to stir overnight at room temperature. Removal of the solvent in vacuo gave 53.1 g (265 mmol) of the product (93%), which was carried on to the next step without further purification.

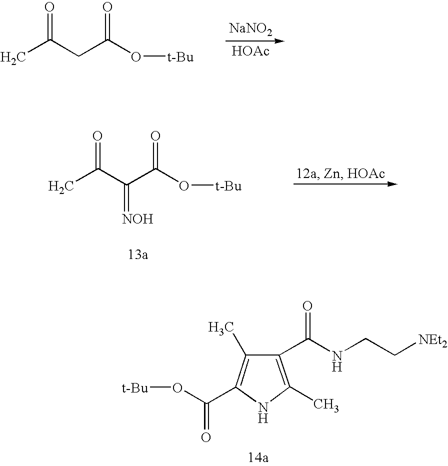

t-Butyl 4-({[2-(diethylamino)ethyl]amino}carbonyl)-3,5-dimethyl-1H-pyrrole-2-carboxylate (14a) via the zinc protocol. t-Butylacetoacetate (60.0 g; 379 mmol) was added to a 1000 mL 3-neck round-bottomed flask equipped with a stopper, addition funnel, and temperature probe. Acetic acid (120 mL) was added to the flask and the mixture cooled to 5° C. A solution of NaNO₂ (27.0 g; 391 mmol) in H₂O (60 mL) was added drop-wise over 45 minutes to the 3-neck flask, keeping the temperature below 10° C. Upon completion of the addition, H₂O (45 mL) was added and the solution was stirred an additional 30 minutes and then allowed to stand at room temperature for 3 h. TLC (SiO₂; 30% ethyl acetate/hexanes) indicated complete consumption of starting material by this time. A pale yellow solution of oxime 13a was observed at this stage. The reaction was assumed to go to completion in quantitative yield (71.0 g; 379 mmol) and the solution was used directly in the next step.

Amide 12a (68.5 g; 342 mmol) was added to a 1000 mL 3-neck round-bottomed flask along with acetic acid (175 mL). The resulting solution was heated to 65° C. and Zn (⅛ quantity of 75.2 g; 1150 mmol) was added to the flask. Once at 65° C., a solution of oxime 13a (⅛ quantity of 66.9 g; 357 mmol) was added. This process was continued until all the zinc and oxime were added. There was a 10-15° C. exotherm between additions; however, the reaction temperature was brought back to 65° C. before the next addition. After the last addition, the reaction mixture was heated to 75° C. and allowed to stir for 1 h. The reaction vessel was then cooled to room temperature and the slurry was filtered through a coarse frit to remove the unreacted zinc. The filtrate was then transferred to a 2000 mL 3-neck round-bottomed flask equipped with an N₂ inlet and overhead stirrer. H₂O (300 mL) was added to the flask and the solution was basified with 50% NaOH solution. Once the pH of the reaction solution reached 9.0, zinc salts started to form; excess NaOH was added until all zinc salts dissolved. The reaction mixture was then split into two batches and each batch was extracted with CH₂Cl₂ (3×250 mL). The organic layers from both batches were combined and washed with brine (300 mL). The organics were concentrated and recrystallized from acetonitrile. The product, pyrrole 14a, was isolated as off white crystals (60.6 g; 181 mmol; 53%). TLC conditions: 86:12:2 CH₂Cl₂/MeOH/NH₄OH. IR (NaBr) 3333, 3284, 3005, 1687, 1601, 1531, 1502, 1434, 1326, 1286 cm⁻¹; ¹H NMR (400 MHz, CDCl₃) δ 8.92 (s, 1 H), 6.43 (s, 1 H), 3.45 (q, J=5.4 Hz, 2 H), 2.62 (t, J=5.9 Hz, 2 H), 2.55 (q, J=7.0 Hz, 4 H), 2.47 (s, 3 H), 2.46 (s, 3 H), 1.55 (s, 9 H), 1.01 (t, J=7.1 Hz, 6 H). ¹³C NMR (100 MHz, CDCl₃) δ 165.7, 161.0, 134.5, 125.7,118.8, 118.3, 80.9, 51.5, 46.5, 36.7, 28.5, 13.4, 11.8, 11.7; HRMS (ES): found, m/z 338.2447 (M+H⁺); C₁₈H₃₁N₃O₃+H requires 338.2443.

t-Butyl 4-({[2-(diethylamino)ethyl]amino}carbonyl)-3,5-dimethyl-1H-pyrrole-2-carboxylate 14a via hydrogenation: t-Butylacetoacetate (30 g; 190 mmol) was added to a 3-neck round-bottomed flask along with acetic acid (30 mL). The mixture was cooled to 0-3 ° C. under N₂ and a solution of NaNO₂ (18.3 g; 265 mmol) dissolved in H₂O (35 mL) was added drop-wise maintaining the temperature below 10° C. Once the addition was complete, the reaction solution was slowly warmed to room temperature. When the reaction was deemed complete by TLC (2 h), the mixture was partitioned between aqueous KCl solution (40 mL) and diethyl ether (50 mL). The aqueous layer was extracted further with diethyl ether (3×25 mL). The combined organics were washed with H₂O (3×35 mL), dried over Na₂SO₄ and concentrated in vacuo to afford oxime 13a as a pale yellow oil which was used in the next step without further purification.

Oxime 13a (20.0 g; 107 mmol) was added to a 500 mL Parr vessel along with 2.0 g of 5% dry Pd/C. Amide 12a (21.4 g; 107 mmol) was dissolved in acetic acid (220 mL) and charged to the Parr bottle. The vessel was purged with N₂ and H₂ and the mixture hydrogenated at 45 psig by heating at 65° C. for 7 h. After this time, the reaction mixture was cooled to room temperature, filtered to remove Pd, and the cake was washed with acetic acid. The filtrate was neutralized with 50% aqueous NaOH. CH₂Cl₂ (500 mL) was added, followed by more 50% aqueous NaOH until the pH of the aqueous phase was 13. The mixture was transferred to a separatory funnel and the layers separated. The aqueous layer was extracted with CH₂Cl₂ (3×350 mL), and the combined organics were washed with H₂O (2×250 mL). The washes were back-extracted with CH₂Cl₂ (250 mL), and the combined organics were concentrated in vacuo. The residue was dissolved in hot CH₃CN and the resulting solution was filtered and cooled. The solids that formed were isolated by filtration to afford 27.7 g (83 mmol; 77%) of pyrrole 14a.

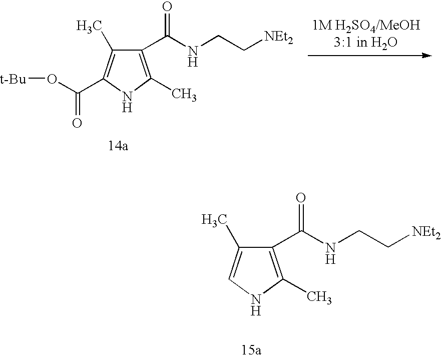

N-[2-(diethylamino)ethyl]-2,4-dimethyl-1H-pyrrole-3-carboxamide (15a). Pyrrole 14a (20.0 g; 60 mmol) was added to a 2000 mL 3-neck round-bottomed flask equipped with an addition funnel, N₂ inlet, and overhead stirrer. A 3:1 mixture of 1 M H₂SO₄/MeOH and H₂O (1200 mL) was added drop-wise (over 15 minutes) to the flask with stirring. Once the addition was complete, the solution was stirred at 65° C. for 3.5 h. The reaction mixture was cooled to 0-5° C. in an ice-water bath. H₂O (200 mL) was added and the solution brought to a pH of 12-14 with 50% NaOH. Some salt formation was observed. The salts were easily filtered off and the filtrate was transferred to a 2000 mL separatory funnel. The aqueous mixture was extracted with CH₂Cl₂ (3×200 mL). The organic phases were combined and washed with H₂O (3×300 mL) followed by a brine wash (300 mL). The organic phases were concentrated to dryness to yield 15a as a light brown oil (14.2 g; 60 mmol; quantitative yield) which was used in the next step without further purification. IR (NaBr) 3246, 2969, 1624, 1577, 1529, 1504 cm⁻¹; ¹H NMR (400 MHz, CDCl₃) δ 8.30 (s, 1 H), 6.44 (s, 1 H), 6.33 (s, 1 H) 3.45 (q, J=5.8 Hz, 2 H), 2.62 (t, J=6.1 Hz, 2 H), 2.56 (q, J=7.1 Hz, 4 H), 2.46 (s, 3 H), 2.23 (s, 3 H), 1.01 (t, J =7.0 Hz, 6 H). ¹³C NMR (100 MHz, CDCl₃) δ 166.7, 132.6, 117.5, 114.3, 51.6, 46.5, 36.6, 13.5, 12.6, 11.7; HRMS (ES): found, m/z 238.1919 (M+H⁺); C₁₃H₂₃N₃O+H requires 238.1921.

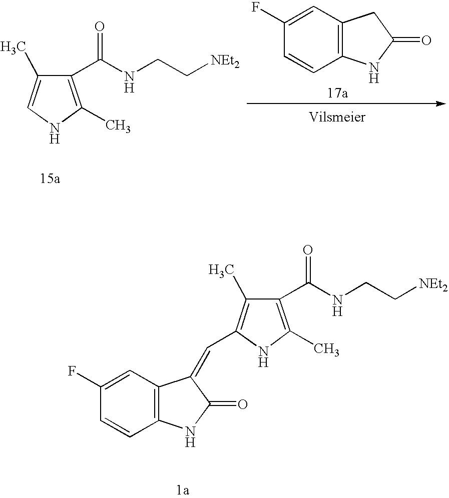

N-[2-(diethylamino)ethyl]-5-[(Z)-(5-fluoro-2-oxo-1,2-dihydro-3H-indol-3-ylidene)methyl]-2,4-dimethyl-1H-pyrrole-3-carboxamide (1a). Chloromethylenedimethylammonium chloride (“Vilsmeier”) (7.8 g; 61 mmol) was added to a 1000 mL 3-neck round-bottomed flask equipped with an addition funnel, N₂ inlet, and overhead stirrer. Acetonitrile (84 mL) was added drop-wise via the addition funnel to the flask. Compound 15a (13.7 g; 58 mmol) was dissolved in acetonitrile (116 mL) and added to the flask through the addition funnel. The amide chloride gradually dissolved and the reaction solution turned dark orange. After 15 minutes, an orange solid precipitated out of solution. The reaction was complete in 40 minutes. The 5-fluorooxindole (a) (9.2 g; 61 mmol) and pulverized KOH (11.9 g; 213 mmol) were added to the reaction mixture and stirring was continued. Acetonitrile (10 mL) was used to help transfer over reagents. An orange solid crashed out immediately. The reaction mixture was stirred at room temperature for 3.5 h, filtered and dried to give 1a (16.9 g; 42 mmol) in 74% yield. IR (NaBr) 3298, 3230, 2968, 1676, 1627, 1590, 1544, 1498, 1334 cm⁻¹; ¹H NMR (400 MHz, CDCl₃) δ 7.75 (dd, J=9.4, 2.5 Hz, 1 H), 7.71 (s, 1 H), 7.43 (t, J=5.6 Hz, 1 H), 6.92 (td, J=9.1, 2.5 Hz, 1 H), 6.84 (dd, J=8.5, 4.6 Hz, 1 H), 3.41-3.36 (m, 2 H), 2.65-2.58 (m, 6 H), 2.47 (s, 3 H), 2.43 (s, 3 H), 1.07 (t, J =7.1 Hz, 6 H); HRMS (ES): found, m/z 399.2204 (M+H⁺); C₂₂H₂₇FN₄O₂+H requires 399.2196.

### Example 2

### Preparation of 5-[(Z)-(5-fluoro-2-oxo-1,2-dihydro-3H-indol-3-ylidene)methyl]-2,4-dimethyl-N-(2-pyrrolidin-1-ylethyl)-1H-pyrrole-3-carboxamide (1b).

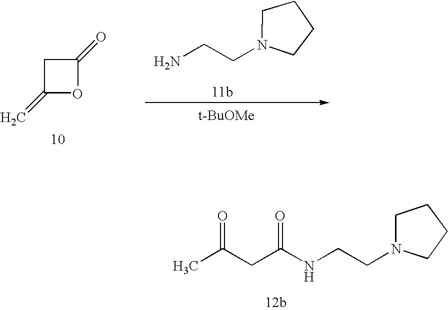

3-oxo-N-(2-pyrrolidin-1-ylethyl)butanamide (12b): Diketene (10) (10.0 g) was added to a 1000 mL 3-neck round-bottomed flask equipped with an addition funnel, N₂ inlet, and overhead stirrer. MTBE (90 mL) was transferred to the flask and the mixture was cooled to 0-5° C. using an ice-water bath. N-(2-Aminoethyl)pyrrolidine (11b) (10.9 g) was added to the reaction drop-wise, maintaining the temperature below 5° C. CH₂Cl₂ (30 mL) was added to the flask to aid in solubility. The ice-water bath was removed and the solution allowed to warm to room temperature and eventually stirred overnight. The product 12b obtained upon concentration of the reaction mixture (21.0 g; 89%) was carried on to the next step without further purification.

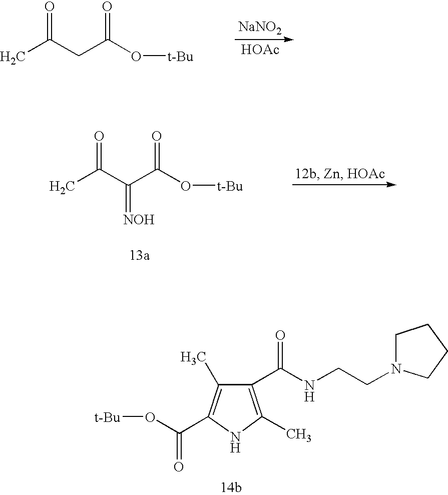

tert-butyl 3,5-dimethyl-4-{[(2-pyrrolidin-1-ylethyl)amino]carbonyl}-1H-pyrrole-2-carboxylate (14b): t-Butyl acetoacetate (12.5 g) was added to a 100 mL 3-neck round-bottomed flask equipped with an addition funnel and temperature probe. Acetic acid (30 mL) was added to the flask and the mixture cooled to 5° C. A solution of NaNO₂ (5.5 g) in H₂O (9 mL) was added drop-wise over 45 minutes to the 3-neck flask, keeping the temperature below 10° C. Upon completion of the addition, the solution was stirred an additional 30 minutes then allowed to stand at room temperature for 3 h. A pale yellow solution of the oxime 13a was observed at this stage. The reaction was assumed to have proceeded in quantitative yield (14.8 g; 79.0 mmol) and the reaction mixture was used directly in the next step.

14b via the zinc protocol: Amide 12b (15.7 g) was added to a 500 mL round-bottomed flask equipped with a temperature probe and condenser. Acetic acid (40 mL) was added to the flask and the mixture placed in a pre-heated oil bath (60° C). Zinc dust (5×4.0 g) was added along with the previously formed oxime 13a (5×10 mL); five sequential additions in all. The first two additions were done quickly, allowing the reaction temperature to reach 90° C. The remaining three additions were completed keeping the temperature between 65 and 75° C. Following the last addition, the reaction temperature was increased to 78° C. and the mixture was stirred for 1 h. The mixture was then cooled to room temperature, poured into 300 mL of H₂O, and filtered through Celite™. The reaction vessel and the Celite™ cake were washed with CH₂Cl₂ (3×20 mL). The filtrate was transferred to a 1000 mL round-bottomed flask and concentrated. The flask was then cooled to 0° C. and the contents were neutralized with NaHCO₃. The aqueous phase was extracted with CH₂Cl₂ (3×150 mL). The combined organics were washed with water (300 mL) and 10% NaOH (100 mL), and concentrated in vacuo. Recrystallization of the crude product 14b from acetonitrile gave the product as white crystals (12.5 g; 48%).

14b via hvdroaenation: Oxime 13a (13.2 g) was added to a 500 mL Parr vessel along with 1.42 g of 5% dry Pd/C. Amide 12b (14.6 g) was dissolved in acetic acid (200 mL) and charged to the Parr bottle. The vessel was purged with N₂ and H₂ and the mixture hydrogenated by heating at 65° C. and 45 psi for 6 h. After this time, the Parr vessel was cooled to room temperature, filtered to remove Pd, and the cake was washed with acetic acid (2×250 mL). The filtrate was neutralized with 50% aqueous NaOH. CH₂Cl₂ (500 mL) was added, and more 50% aqueous NaOH until the pH of the aqueous phase was 13. The mixture was transferred to a separatory funnel and the layers separated. The aqueous layer was extracted with CH₂Cl₂ (3×250 mL), and the combined organics were washed with H₂O (3×250 mL). The washes were back-extracted with CH₂Cl₂ (250 mL), and the combined organics were concentrated in vacuo to afford 21.6 g (92%) of the product 14b as a yellow solid. Recrystallization from CH₃CN/hexanes gave 11.3 g (48%) of the product as a white powder. ¹H NMR (400 MHz, CDCl₃) δ 8.99 (s, 1 H), 6.46 (s, 1 H), 3.54 (q, J=5.5 Hz, 2 H), 2.73 (t, J=5.9 Hz, 2 H), 2.60 (m, 4 H), 2.46 (s, 6 H), 1.79 (m, 4 H), 1.58 (s, 9 H). ¹³C NMR (400 MHz, CDCl₃) δ 165.8, 161.1, 134.2, 125.3, 118.8, 118.5, 80.9, 54.4, 53.6, 37.8, 28.5, 23.5, 13.2, 11.6.

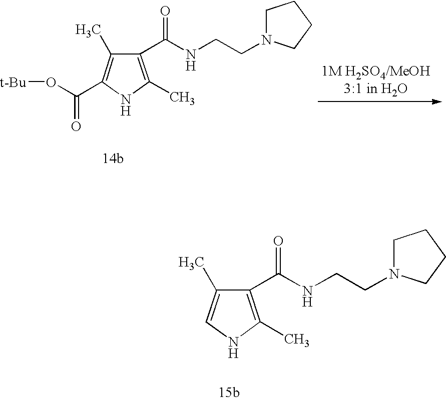

2,4-dimethyl-N-(2-pyrrolidin-1-ylethyl)-1H-pyrrole-3-carboxamide (15b): 14b (5.0 g) was added to a 1000 mL 3-neck round-bottomed flask equipped with an addition funnel, N₂ inlet, and overhead stirrer. A 1M H₂SO₄/MeOH 3:1 in H₂O solution (300 mL) was added drop-wise (over 15 minutes) to the flask with stirring. Once the addition was complete the solution was stirred at 65° C. for 3.5 h. The reaction mixture was cooled to room temperature, then to 0-5° C. in an ice-water bath. H₂O (500 mL) was added and the solution brought to a pH of 12-14 with 50% NaOH solution. The aqueous mixture was extracted with CH₂Cl₂ (3×200 mL). The organics were combined and washed with H₂O (300 mL). The organics were concentrated to dryness yielding 15b as a light brown solid (4.2 g; quantitative yield). ¹H NMR (400 MHz, CDCl₃) δ 8.30 (s, 1 H), 6.42 (s, 1 H), 6,36 (s, 1 H) 3.54 (q, J =5.6 Hz, 2 H), 2.72 (t, J =6.1 Hz, 2 H), 2.60 (m, 4 H), 2.47 (s, 3 H), 2.24 (s, 3 H), 1.80 (m, 4 H). ¹³C NMR (400 MHz, CDCl₃) δ 166.7, 132.3, 117.4, 114.3, 114.2, 54.4, 53.7, 37.7, 23.6, 13.4, 12.3.

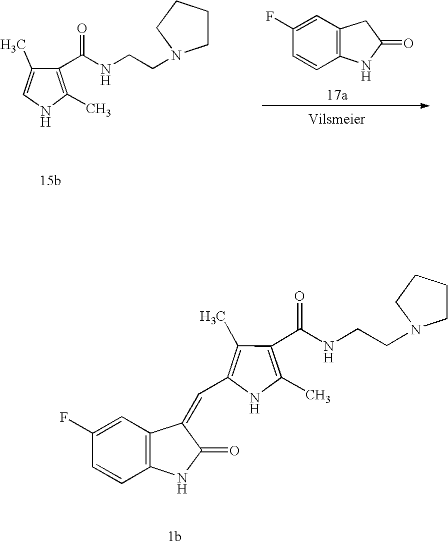

5-[(Z)-(5-fluoro-2-oxo-1,2-dihydro-3indol-3-ylidene)methyl]-2,4-dimethyl-N-(2-pyrrolidin-1-ylethyl)-1H-pyrrole-3-carboxamide (1b): Amide chloride (1.2 g) and acetonitrile (14 mL) were added to a 250 mL 3-neck round-bottomed flask equipped with a N₂ inlet and overhead stirrer. 15b (2.0 g) was taken up in acetonitrile (20 mL) and added to the flask. After 10 minutes, a brown solid precipitated out of solution and the reaction was allowed to stir for 1 h. Oxindole 17a (1.35 g) and KOH (0.95 g) were added to the reaction mixture and stirring was continued. Acetonitrile (3 mL) was used to help transfer over reagents and the reaction mixture was stirred at room temperature overnight. The reaction solution was distilled down to a thick paste and H₂O (30 mL) and H₃PO₄ (1.2 g) were added. The solution was heated at 35° C. for 1 h. The temperature was raised to 55° C. and ethanol (40 mL) was slowly added. Once the addition was complete, the mixture was cooled to 0° C. Very little product precipitated out of solution at this point, so the mixture was distilled to a lower volume (approx. 20 mL) to induce precipitation. Upon cooling, the product precipitated out as a yellow solid, the phosphoric acid salt of 1b, which was isolated by filtration (2.47 g; 59%).

We claim:

1. A method of preparing a compound of formula 1 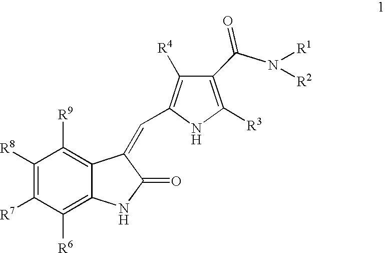
wherein:
R¹ is —(CH₂)ₘR¹⁰, and one or more hydrogens in the —(CH₂)ₘ groups is optionally substituted by —OH;
R² is H or C₁₋₁₂ alkyl;
optionally, R¹ and R², together with the nitrogen to which they are attached, can join to form a 5, 6 or 7-membered heterocyclic group optionally containing an additional N, O or S ring atom;
each R³ and R⁴ is independently C₁₋₁₂ alkyl;
each R⁶, R⁷, R⁸ and R⁹ is independently selected from the group consisting of hydrogen, C₁₋₁₂ alkyl, C₁₋₁₂ alkoxy, C₃₋₁₂ cycloalkyl, C₆₋₁₂ aryl, C₂₋₁₂ heterocyclic group containing 1 to 3 atoms selected from N, S and O, C₆₋₁₂ aryloxy, C₆₋₁₂ alkaryl, C₆₋₁₂ alkaryloxy, halogen, trihalomethyl, —S(O)R¹⁶, —SO₂NR¹⁶R¹⁷, —SO₃R¹⁶, —SR¹⁶, —NO₂, —NR¹⁶R¹⁷, —OH, —CN, —C(O)R¹⁶, —OC(O)R¹⁶, —NHC(O)R¹⁶, —(CH₂)ₙCO₂R¹⁶, and —CONR¹⁶R¹⁷;
R¹⁰ is selected from the group consisting of —NR¹¹R¹², —OH, —C(O)R¹³, C₆₋₁₂ aryl, C₆₋₁₂ alkaryl, C⁶⁻¹² aryloxy, C⁶⁻¹² alkaryloxy, C₁₋₁₂ alkoxy, —N⁺(O⁻)R¹¹R¹², —NHC(O)R¹⁴, and C₂₋₁₂ heterocyclic group containing 1 to 3 atoms selected from N, S and O;
R¹¹ and R¹² are independently selected from the group consisting of hydrogen, C₁₋₁₂ alkyl, C₁₋₁₂ cyanoalkyl, C₃₋₁₂ cycloalkyl, C₆₋₁₂ aryl, and C₂₋₁₂ heterocyclic group containing 1 to 3 atoms selected from N, S or O; or R¹¹ and R¹² may be combined to form a four-, five- or six-membered heterocyclic group optionally containing 1 to 3 atoms selected from N, O, or S in addition to the nitrogen atom to which R¹¹ and R¹² are bound, provided that the heterocyclic group formed by R¹¹ and R¹² may optionally be substituted by one or more R¹⁵ groups;
R¹³ is selected from the group consisting of —OH, C₁₋₁₂ alkyl, C₆₋₁₂ aryl, C₁₋₁₂ alkoxy, C₆₋₁₂ alkaryl, C₆₋₁₂ alkaryloxy and C₆₋₁₂ aryloxy;
R¹⁴ is selected from the group consisting of C₁₋₁₂ alkyl, C₁₋₁₂ haloalkyl, and C₆₋₁₂ aralkyl;
R¹⁵ is C₁₋₁₂ alkyl, C₁₋₁₂ cyanoalkyl, C₅₋₁₂ cycloalkyl, C₆₋₁₂ aryl or C₂₋₁₂ heterocyclic group containing 1 to 3 atoms selected from N, S and O;
R¹⁶ and R¹⁷ are independently selected from the group consisting of hydrogen, C₁₋₁₂ alkyl, C₁₋₁₂ cyanoalkyl, C₅₋₁₂ cycloalkyl, C₆₋₁₂ aryl, C₂₋₁₂ heterocyclic group containing 1 to 3 atoms selected from N, S and O, or in the group —NR¹⁶R¹⁷, R¹⁶ and R¹⁷ may be combined to form a four-, five- or six-membered heterocyclic group optionally containing 1 to 3 atoms selected from N, O, or S in addition to the nitrogen atom to which R¹⁶ and R¹⁷ are bound;
m is 0, 1, 2, 3 or 4; and
n is 0, 1 or 2;
the method comprising reacting a compound of formula 15 with a compound of formula 17 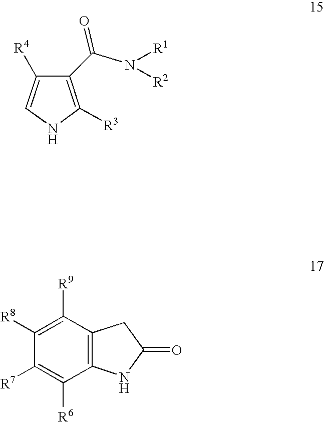
and a formylating reagent to form the compound of formula 1.

2. The method of claim 1, wherein R¹⁰ is —NR¹¹R¹² and R¹¹ and R¹² are independently H or C₁₋₄ alkyl.

3. The method of claim 1, wherein R¹⁰ is —NR¹¹R¹², where R¹¹ and R¹², together with the nitrogen atom to which they are bound, are combined to form a five or six-membered heterocyclic group optionally containing an additional N, O or S ring atom.

4. The method of claim 1, wherein m is 2, 3 or 4 and R¹⁰ is a heterocyclic group selected from 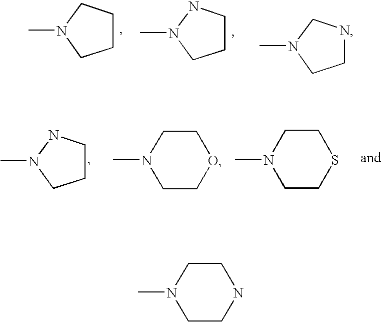
optionally substituted by one or more R¹⁵ groups.

5. The method of claim 1, wherein the compound of formula 1 is selected from the group consisting of

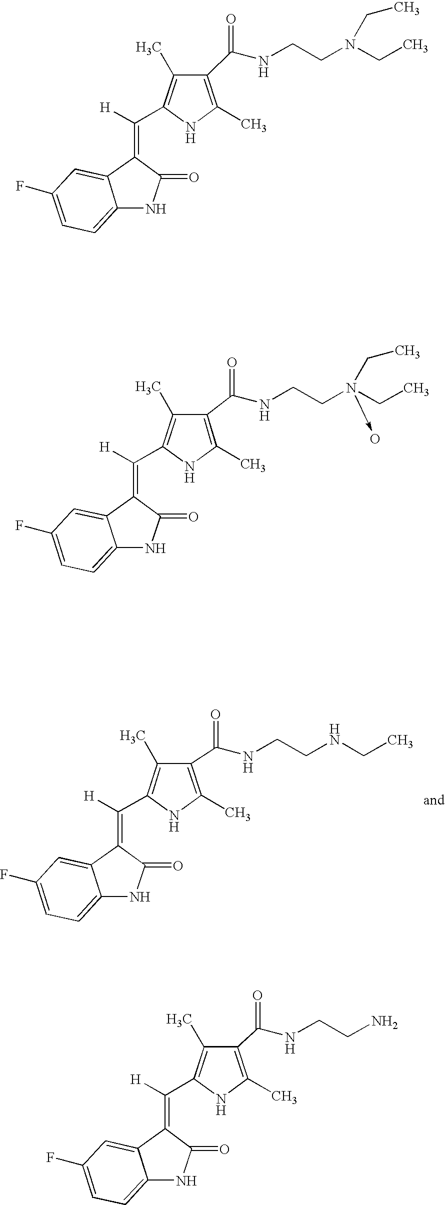

6. The method of claim 1, wherein the compound of formula 1 is selected from the group consisting of:

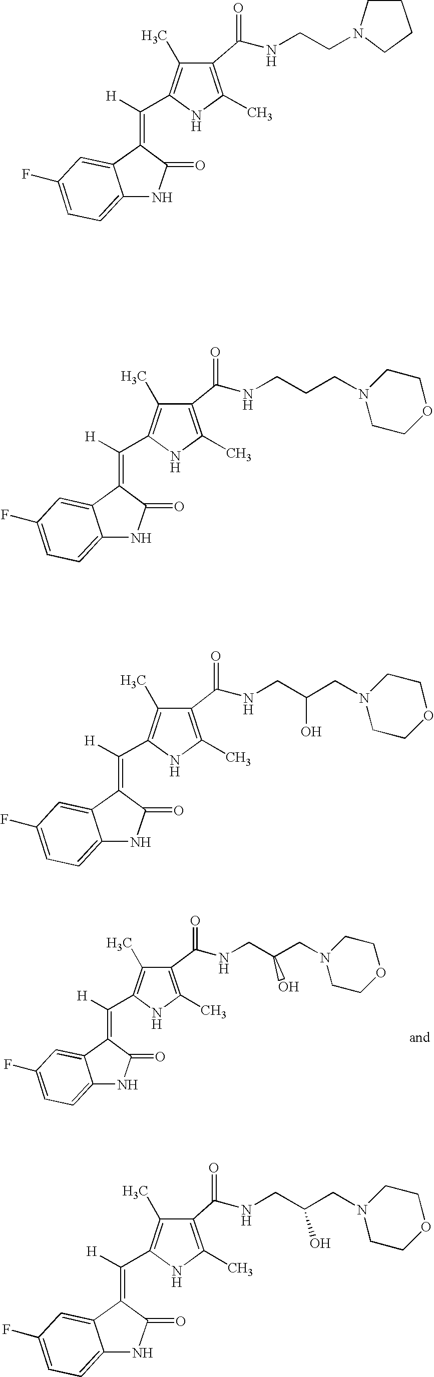

7. The method of claim 1, wherein the formylating agent is a halo-substituted iminium salt.

8. The method of claim 7, wherein the formylating agent is chloromethylenedimethylammonium chloride.

9. The method of claim 1, wherein the formylating agent is formed in situ from DMF in POCl₃.

10. The method of claim 1, wherein the formylating agent is a trialkylorthoformate.

11. The method of claim 1, wherein the step of reacting the compound of formula 15 with the compound of formula 17 and the formylating agent is carried out by (i) reacting the compound of formula 15 with the formylating agent to form an intermediate; and (ii) reacting the intermediate with the compound of formula 17 to form the compound of formula 1.

12. The method of claim 11, wherein the formylating agent is a compound of formula A-L, where L is a halogen or a leaving group, A is a formyl group or a group that can be hydrolyzed to a formyl group, and the intermediate is a compound of formula 16

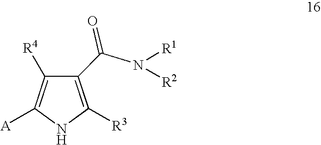

13. The method of claim 12, wherein the compound of formula A-L is a halo-substituted iminium salt.

14. The method of claim 12, wherein the compound of formula A-L is chloromethylenedimethylammonium chloride and the intermediate is a compound of formula:

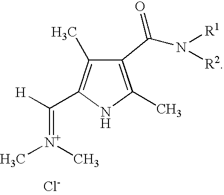

15. The method of claim 12, wherein the compound of formula A-L is a trialkylorthoformate, and the intermediate is a compound of formula: 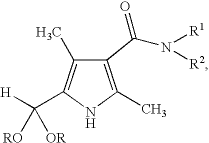
wherein each R group is independently C₁₋₁₂ alkyl.

16. The method of claim 1, wherein the compound of formula 15 is formed by decarboxylating a compound of formula 14 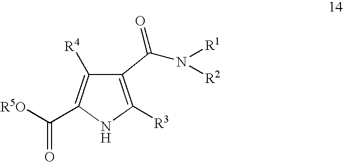
wherein R₅ is a C₁₋₁₂ alkyl group to form the compound of formula 15.

17. The method of claim 16, wherein the compound of formula 14 is formed by reacting a compound of formula 12 with a compound of formula 13 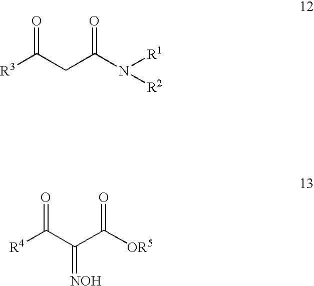
under pyrrole formation conditions to form the compound of formula 14.

18. The method of claim 17, wherein the pyrrole formation conditions comprises carrying out the reaction in the presence of zinc and acetic acid.

19. The method of claim 17, wherein the pyrrole formation conditions comprises carrying out the reaction in the presence of H₂ and a hydrogenation catalyst.

20. A method of preparing a pyrrole of formula 14 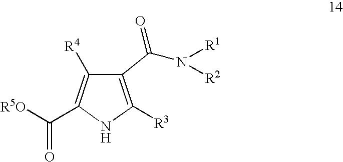
wherein:
R¹ is —(CH₂)ₘR¹⁰, and one or more hydrogens in the —(CH₂)ₘ groups is optionally substituted by —OH;
R² is H or C₁₋₁₂ alkyl;
optionally, R¹ and R², together with the nitrogen to which they are attached, can join to form a 5, 6 or 7-membered heterocyclic group optionally containing an additional N, O or S ring atom;
each R³ and R⁴ is independently C₁₋₁₂ alkyl;
R₅ is C₁₋₁₂ alkyl;
R¹⁰ is selected from the group consisting of —NR¹¹R¹², —OH, —C(O)R¹³, C₆₋₁₂ aryl, C₆₋₁₂ alkaryl, C₆₋₁₂ aryloxy, C₆₋₁₂ alkaryloxy, C₁₋₁₂ alkoxy, —N⁺(O⁻)R¹¹R¹², —NHC(O)R¹⁴, and C₂₋₁₂ heterocyclic group containing 1 to 3 atoms selected from N, S and O;
R¹¹ and R¹² are independently selected from the group consisting of hydrogen, C₁₋₁₂ alkyl, C₁₋₁₂ cyanoalkyl, C₃₋₁₂ cycloalkyl, C₆₋₁₂ aryl, and C₂₋₁₂ heterocyclic group containing 1 to 3 atoms selected from N, S or O; or R¹¹ and R¹² may be combined to form a four-, five- or six-membered heterocyclic group optionally containing 1 to 3 atoms selected from N, O, or S in addition to the nitrogen atom to which R¹¹ and R¹² are bound, provided that the heterocyclic group formed by R¹¹ and R¹² may optionally be substituted by one or more R¹⁵ groups;
R¹³ is selected from the group consisting of —OH, C₁₋₁₂ alkyl, C₆₋₁₂ aryl, C₁₋₁₂ alkoxy, C₆₋₁₂ alkaryl, C₆₋₁₂ alkaryloxy and C₆₋₁₂ aryloxy;
R¹⁴ is selected from the group consisting of C₁₋₁₂ alkyl, C₁₋₁₂ haloalkyl, and C₆₋₁₂ aralkyl;
R¹⁵ is C₁₋₁₂ alkyl, C₁₋₁₂ cyanoalkyl, C₅₋₁₂ cycloalkyl, C₆₋₁₂ aryl or C₂₋₁₂ heterocyclic group containing 1 to 3 atoms selected from N, S and O; and
m is 0, 1, 2, 3 or 4;
the method comprising reacting a compound of formula 12 with a compound of formula 13 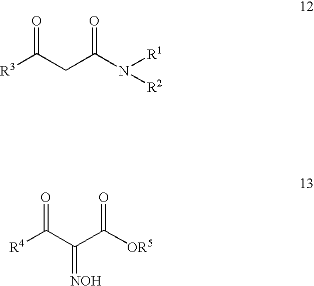
under pyrrole formation conditions to form the compound of formula 14.

21. The method of claim 20, wherein R¹⁰ is —NR¹¹R¹² and R¹¹ and R¹² are independently H or C₁₋₄ alkyl.

22. The method of claim 20, wherein R¹⁰ is —NR¹¹R¹², where R¹¹ and R¹², together with the nitrogen atom to which they are bound, are combined to form a five or six-membered heterocyclic group optionally containing an additional N, O or S ring atom.

23. The method of claim 20, wherein m is 2, 3 or 4 and R¹⁰ is a heterocyclic group selected from 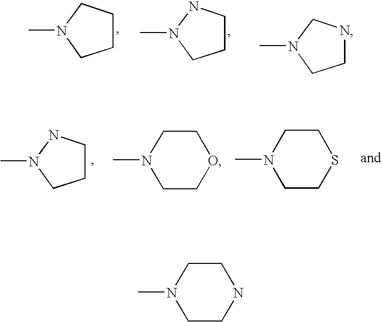
optionally substituted by one or more R¹⁵ groups.

24. The method of claim 20, wherein the compound of formula 14 is selected from the group consisting of

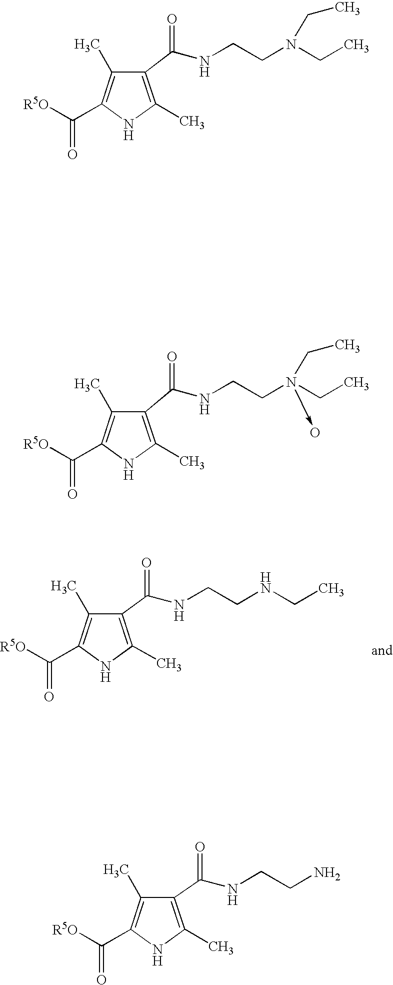

25. The method of claim 20, wherein the compound of formula 14 is selected from the group consisting of:

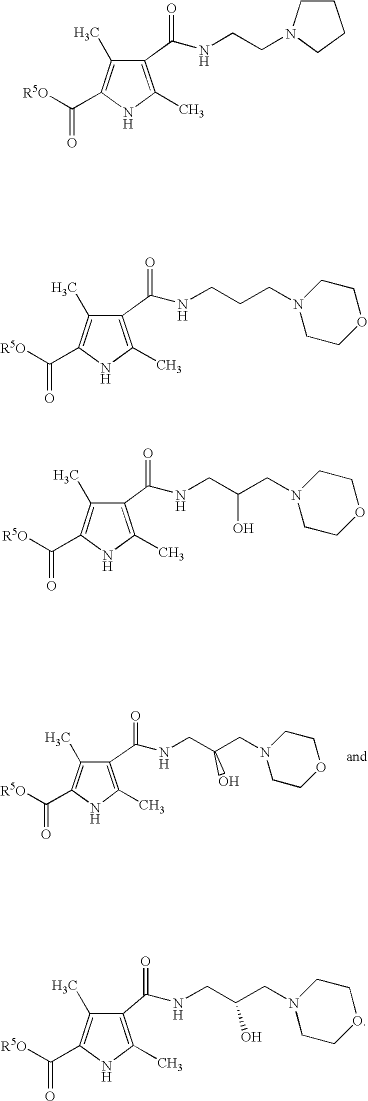

26. The method of claim 20, wherein the pyrrole formation conditions comprises carrying out the reaction in the presence of zinc and acetic acid.

27. The method of claim 20, wherein the pyrrole formation conditions comprises carrying out the reaction in the presence of H₂ and a hydrogenation catalyst.

28. The method of claim 20, further comprising decarboxylating the compound of formula 14 to form a compound of formula 15

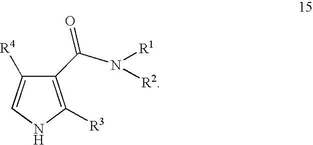

29. The method of claim 28, further comprising reacting the compound of formula 15 with a compound of formula 17 and a formylating agent to form a compound of formula 1 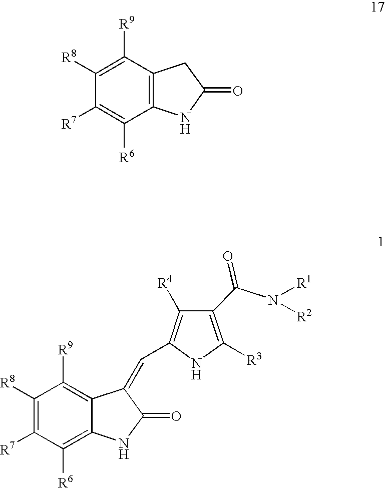
wherein
each R⁶, R⁷, R⁸ and R⁹ is independently selected from the group consisting of hydrogen, C₁₋₁₂ alkyl, C₁₋₁₂ alkoxy, C₃₋₁₂ cycloalkyl, C₆₋₁₂ aryl, C₂₋₁₂ heterocyclic group containing 1 to 3 atoms selected from N, S and O, C₆₋₁₂ aryloxy, C₆₋₁₂ alkaryl, C₆₋₁₂ alkaryloxy, halogen, trihalomethyl, —S(O)R¹⁶, —SO₂NR¹⁶R¹⁷, —SO₃R¹⁶, —SR¹⁶, —NO₂, —NR¹⁶R¹⁷, —OH, —CN, C(O)R¹⁶, —C(O)R¹⁶, —NHC(O)R¹⁶, —(CH₂)ₙCO₂R¹⁶, and —CONR¹⁶R¹⁷;
R¹⁶ and R¹⁷ are independently selected from the group consisting of hydrogen, C₁₋₁₂ alkyl, C₁₋₁₂ cyanoalkyl, C₅₋₁₂ cycloalkyl, C₁₋₁₂ aryl, C₂₋₁₂ heterocyclic group containing 1 to 3 atoms selected from N, S and O, or in the group —NR¹⁶R¹⁷, R¹⁶ and R¹⁷ may be combined to form a four-, five- or six-membered heterocyclic group optionally containing 1 to 3 atoms selected from N, O, or S in addition to the nitrogen atom to which R¹⁶ and R¹⁷ are bound; and
n is 0, 1 or 2.

30. The method of claim 29, wherein the formylating agent is a halo-substituted iminium salt.

31. The method of claim 30, wherein the formylating agent is chloromethylenedimethylammonium chloride.

32. The method of claim 29, wherein the formylating agent is formed in situ from DMF in POCl₃.

33. The method of claim 29, wherein the formylating agent is a trialkylorthoformate.

34. The method of claim 29, wherein the step of reacting the compound of formula 15 with the compound of formula 17 and the formylating agent is carried out by (i) reacting the compound of formula 15 with the formylating agent to form an intermediate; and (ii) reacting the intermediate with the compound of formula 17 to form the compound of formula 1.

35. The method of claim 34, wherein the formylating agent is a compound of formula A-L, where L is a halogen or a leaving group, A is a formyl group or a group that can be hydrolyzed to a formyl group, and the intermediate is a compound of formula 16

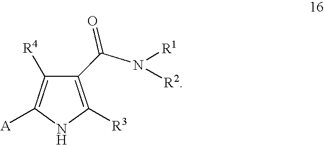

36. The method of claim 34, wherein the compound of formula A-L is a halo-substituted iminium salt.

37. The method of claim 36, wherein the compound of formula A-L is chloromethylenedimethylammonium chloride and the intermediate is a compound of formula:

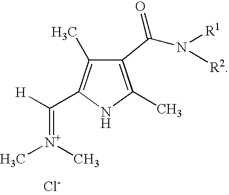

38. The method of claim 29, wherein the compound of formula A-L is a trialkylorthoformate, and the intermediate is a compound of formula: 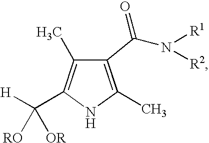
wherein each R group is independently C₁₋₁₂ alkyl.

39. The method of claim 29, wherein the compound of formula 1 is selected from the group consisting of

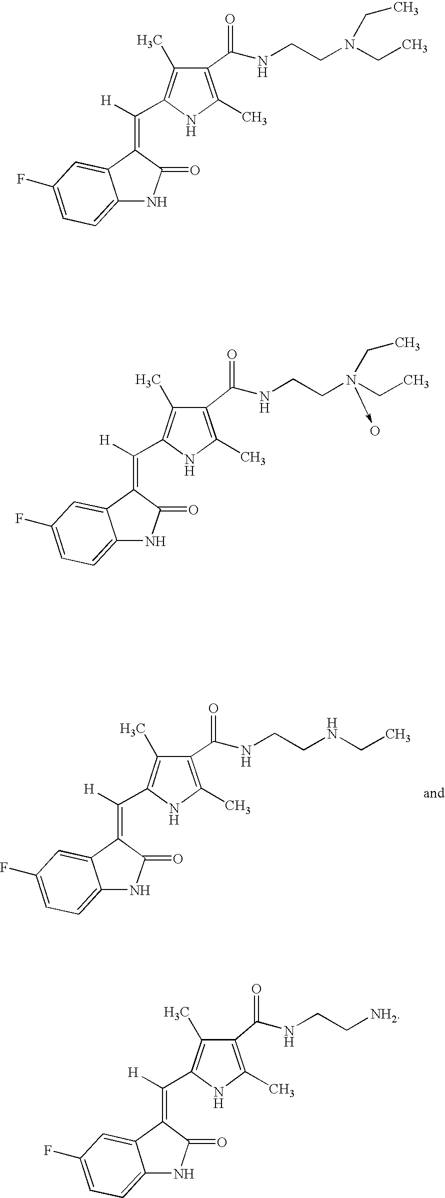

40. The method of claim 29, wherein the compound of formula 1 is selected from the group consisting of:

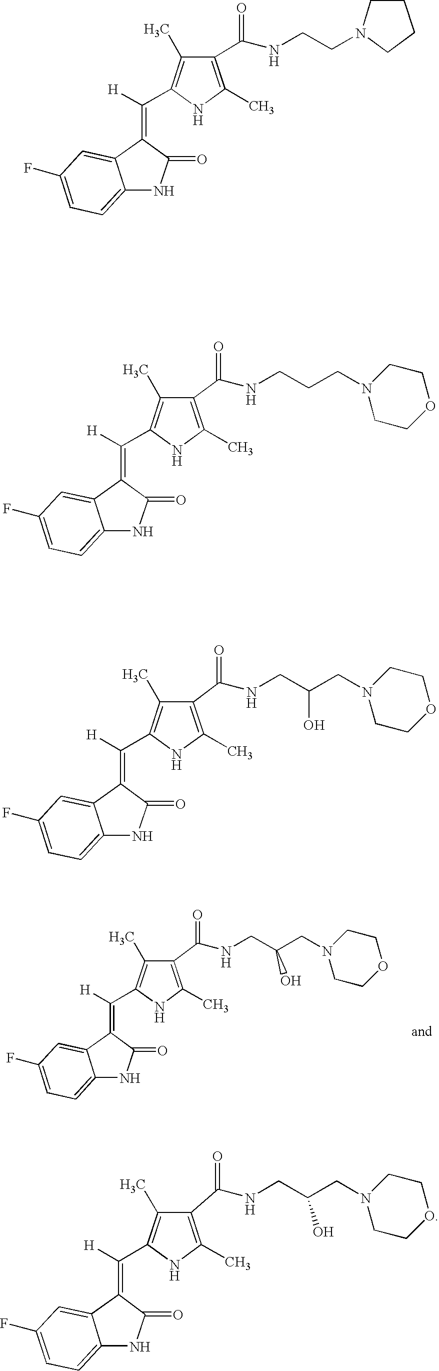

41. The method of claim 20, further comprising reacting a compound of formula 14 with a trialkylorthoformate, HC(OR)₃ to form a compound of formula 24. 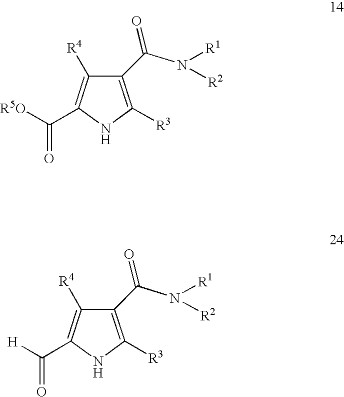
wherein R₅ is a C₁₋₁₂ alkyl group, and each R is independently C₁₋₁₂ alkyl.

42. The method of claim 41, further comprising reacting the compound of formula 24 with a compound of formula 17 to form a compound of formula 1 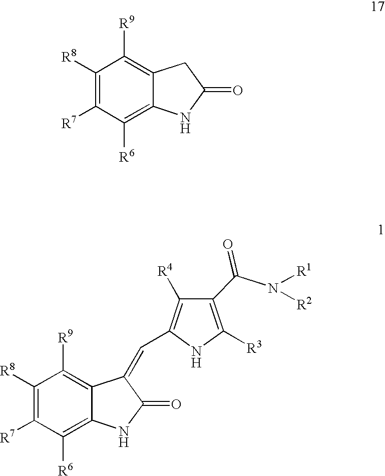
wherein
each R⁶, R⁷, R⁸ and R⁹ is independently selected from the group consisting of hydrogen, C₁₋₁₂ alkyl, C₁₋₁₂ alkoxy, C₃₋₁₂ cycloalkyl, C₆₋₁₂ aryl, C₂₋₁₂ heterocyclic group containing 1 to 3 atoms selected from N, S and O, C₆₋₁₂ aryloxy, C₆₋₁₂ alkaryl, C₆₋₁₂ alkaryloxy, halogen, trihalomethyl, —S(O)R¹⁶, —SO₂NR¹⁶R¹⁷, —SO₃R¹⁶, —SR¹⁶, —NO₂, —NR¹⁶R¹⁷, —OH, —CN, —C(O)R¹⁶, —OC(O)R¹⁶, —NHC(O)R¹⁶, —(CH₂)ₙCO₂R¹⁶, and —CONR¹⁶R¹⁷;
R¹⁶ and R¹⁷ are independently selected from the group consisting of hydrogen, C₁₋₁₂ alkyl, C₁₋₁₂ cyanoalkyl, C₅₋₁₂ cycloalkyl, C₆₋₁₂ aryl, C₂₋₁₂ heterocyclic group containing 1 to 3 atoms selected from N, S and O, or in the group —NR¹⁶R¹⁷, R¹⁶ and R¹⁷ may be combined to form a four-, five- or six-membered heterocyclic group optionally containing 1 to 3 atoms selected from N, O, or S in addition to the nitrogen atom to which R¹⁶ and R¹⁷ are bound; and
n is 0, 1 or 2.

43. The method of claim 42, wherein the compound of formula 1 is selected from the group consisting of

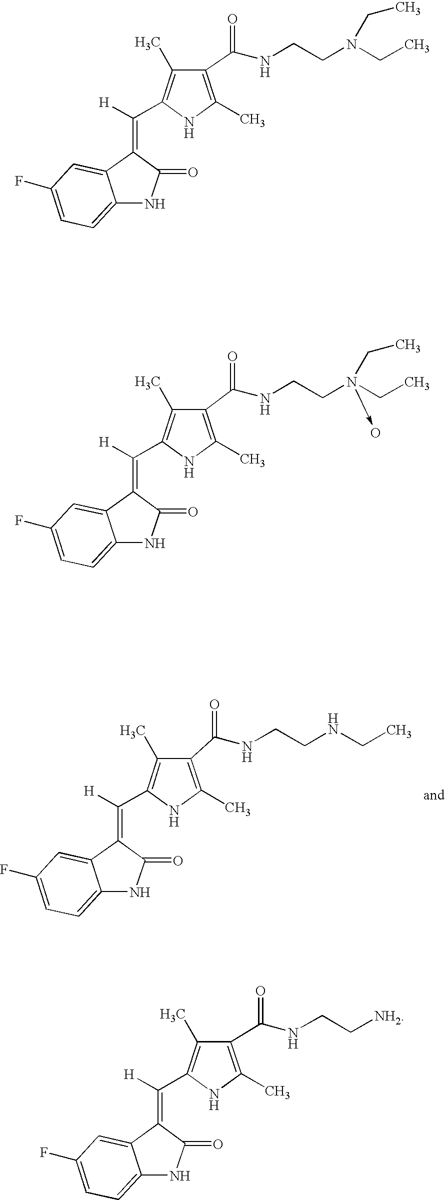

44. The method of claim 42, wherein the compound of formula 1 is selected from the group consisting of:

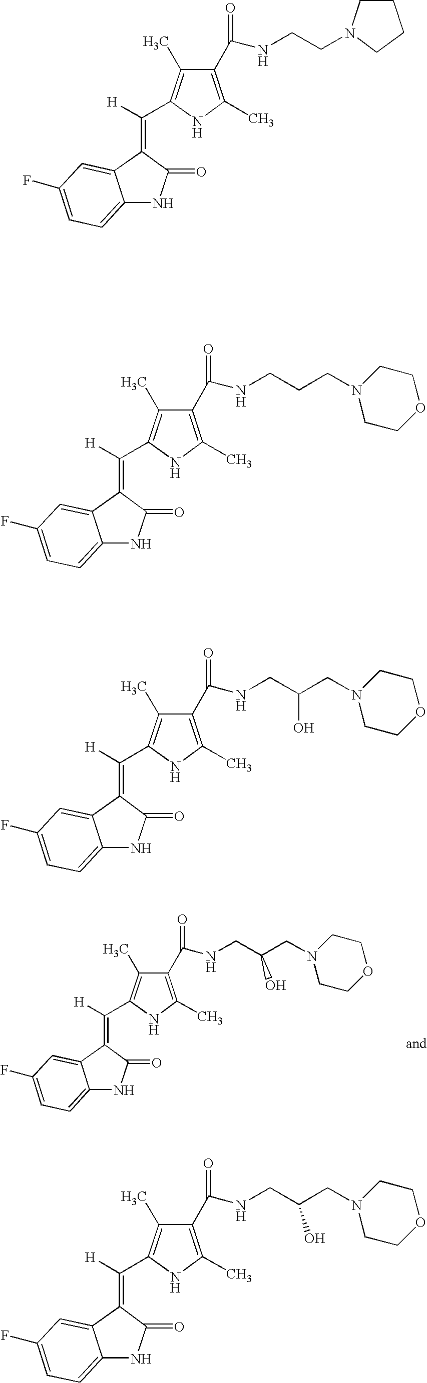

45. A method of preparing a compound of formula 1 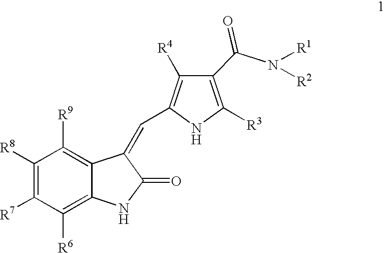
wherein:
R¹ is —(CH₂)ₘR¹⁰, and one or more hydrogens in the —(CH₂)ₘ groups is optionally substituted by —OH;
R² is H or C₁₋₁₂ alkyl;
optionally, R¹ and R², together with the nitrogen to which they are attached, can join to form a 5, 6 or 7-membered heterocyclic group optionally containing an additional N, O or S ring atom;
each R³ and R⁴ is independently C₁₋₁₂ alkyl;
each R⁶, R⁷, R⁸ and R⁹ is independently selected from the group consisting of hydrogen, C₁₋₁₂ alkyl, C₁₋₁₂ alkoxy, C₃₋₁₂ cycloalkyl, C₁₋₁₂ aryl, C₂₋₁₂ heterocyclic group containing 1 to 3 atoms selected from N, S and O, C₆₋₁₂ aryloxy, C₆₋₁₂ alkaryl, C₆₋₁₂ alkaryloxy, halogen, trihalomethyl, —S(O)R¹⁶, —SO₂NR¹⁶R¹⁷, —SO₃R¹⁶, —SR¹⁶, —NO₂, —NR¹⁶R¹⁷, —OH, —CN, —C(O)R¹⁶, —C(O)R¹⁶, —NHC(O)R¹⁶, —(CH₂)ₙCO₂R¹⁶, and —CONR¹⁶R¹⁷;
R¹⁰ is selected from the group consisting of —NR¹¹R¹², —OH, —C(O)R¹³, C₆₋₁₂ aryl, C₆₋₁₂ alkaryl, C⁶⁻¹² aryloxy, C⁶⁻¹² alkaryloxy, C₁₋₁₂ alkoxy, —N⁺(O⁻)R¹¹R¹², —NHC(O)R¹⁴, and C₂₋₁₂ heterocyclic group containing 1 to 3 atoms selected from N, S and O;
R¹¹ and R¹² are independently selected from the group consisting of hydrogen, C₁₋₁₂ alkyl, C₁₋₁₂ cyanoalkyl, C₃₋₁₂ cycloalkyl, C₆₋₁₂ aryl, and C₂₋₁₂ heterocyclic group containing 1 to 3 atoms selected from N, S or O; or R¹¹ and R¹² may be combined to form a four-, five- or six-membered heterocyclic group optionally containing 1 to 3 atoms selected from N, O, or S in addition to the nitrogen atom to which R¹¹ and R¹² are bound, provided that the heterocyclic group formed by R¹¹ and R¹² may optionally be substituted by one or more R¹⁵ groups;
R¹³ is selected from the group consisting of —OH, C₁₋₁₂ alkyl, C₆₋₁₂ aryl, C₁₋₁₂ alkoxy, C₆₋₁₂ alkaryl, C₆₋₁₂ alkaryloxy and C₆₋₁₂ aryloxy;
R¹⁴ is selected from the group consisting of C₁₋₁₂ alkyl, C₁₋₁₂ haloalkyl, and C₆₋₁₂ aralkyl;
R¹⁵ is C₁₋₁₂ alkyl, C₁₋₁₂ cyanoalkyl, C₅₋₁₂ cycloalkyl, C₆₋₁₂ aryl or C₂₋₁₂ heterocyclic group containing 1 to 3 atoms selected from N, S and O;
R¹⁶ and R¹⁷ are independently selected from the group consisting of hydrogen, C₁₋₁₂ alkyl, C₁₋₁₂ cyanoalkyl, C₅₋₁₂ cycloalkyl, C₆₋₁₂ aryl, C₂₋₁₂ heterocyclic group containing 1 to 3 atoms selected from N, S and O, or in the group —NR¹⁶R¹⁷, R¹⁶ and R¹⁷ may be combined to form a four-, five- or six-membered heterocyclic group optionally containing 1 to 3 atoms selected from N, O, or S in addition to the nitrogen atom to which R¹⁶ and R¹⁷ are bound;
m is 0, 1, 2, 3 or 4; and
n is 0, 1 or 2;
the method comprising reacting a compound of formula 24 with a compound of formula 17 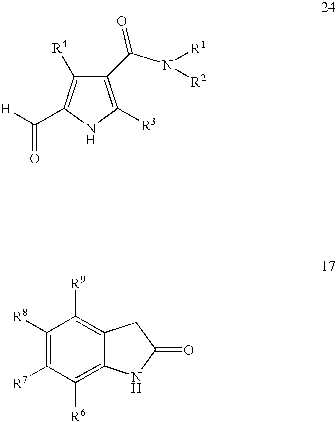
to form the compound of formula 1.

46. The method of claim 45, wherein the compound of claim 24 is formed by reacting a compound of formula 14 with a trialkylorthoformate, HC(OR)₃ 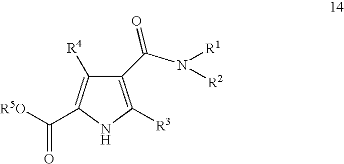
wherein R₅ is a C₁₋₁₂ alkyl group, and each R is independently C₁₋₁₂ alkyl.
